(19) United States
(12) Patent Application Publication
(10) Pub. No.: US 2026/0199589 A1
(43) Pub. Date: Jul. 16, 2026

(54) PLUNGER ROD RELEASABLY ATTACHABLE TO PLUNGER HEAD IN A PUMP DEVICE

(71) Applicant: NeuroDerm, Ltd., Rehovot (IL)

(72) Inventors: Lior Bar Chen, Ganei Tal (IL); Asaf Hazan, Tel-Aviv (IL)

(21) Appl. No.: 19/134,999

(22) PCT Filed: Nov. 23, 2023
(86) PCT No.: PCT/IL2023/051208

§ 371 Date: Jun. 2, 2025

Related U.S. Application Data
(60) Provisional application No. 63/429,781, filed on Dec. 2, 2022.

(51) Int. Cl. A61M 5/142; A61M 5/145; A61M 5/315

(52) U.S. Cl. CPC A61M 5/14216; A61M 5/14566; A61M 5/31515; A61M 5/31583; A61M 2005/14268; A61M 2005/14573; A61M 2005/31588

(57) ABSTRACT

A mechanism for a pump device includes a rotatable earn body that is axially affixed to a distal end of a plunger rod stowable In a pump device and rotatable around the plunger rod. The earn body includes cam surfaces to cammingly co-act with stationary cam pins in the plunger head. Co-action between the cam surfaces of the cam body and the cam pins causes the cam body to rotate in the plunger head to, and from, an engagement angular position in which the cam body and the plunger head are engaged, to a. disengagement, angular position in winch the cam body and the plunger head are disengaged, via an alternating series of push-pull angular positions configured for alternately pushing and pulling the plunger head in the syringe. Rotating the cam body from one angular position to another is implemented by means of reciprocating axial movement of the plunger rod.

### CROSS-REFERENCE TO RELATED APPLICATIONS

This application claims priority to and the benefit of U.S. Provisional Ser. No. 63/429,781 , titled “Plunger Rod Releasably Attachable to Plunger Head in a Pump Device,” which was filed on Dec. 2, 2022, the disclosure of which is herein incorporated by reference in its entirety.

### FIELD OF INVENTION

The present invention generally relates to systems and methods for coupling a disposable reservoir (e.g., syringe) of a drug delivery device (e.g., pump device) to a reusable part (“RP”) of the drug delivery device. More specifically, the present invention relates to a mechanism, plunger rod and plunger head structures that enable to releasably engage the plunger rod with the plunger head, and to effect a series of alternating push-pull angular positions for alternately pushing and pulling the plunger head in the disposable reservoir between engagement of the plunger rod and plunger head and disengagement thereof.

### BACKGROUND

Some liquid drug delivery systems are two-part systems. Such systems typically include a reusable part and a disposable part (e.g., disposable syringe). The reusable part typically includes, among other things, an electric motor and a gear system that is driven by the electric motor. The disposable part typically includes a reservoir (e.g., syringe) and a gear-driven plunger means to expel drug from the medicament reservoir.

In conventional drug delivery systems, the plunger means includes a plunger rod (a form of a “leadscrew”) and a plunger head (a form of a “piston”), and the plunger rod is usually inseparable from the plunger head. In addition, such a plunger means is usually inseparable from the reservoir itself, so, using such a plunger means has drawbacks. For example, the disposable reservoir is disposed of after use together with the plunger rod and plunger head, which is wasteful, hence uneconomic. In addition, since the plunger rod (leadscrew) is conventionally an integral part of the disposable reservoir, coupling between the disposable reservoir and the reusable part oftentimes results in degraded accuracy of the medicament delivered due to some uncertainty with regard to the precise location, or axial position, of the plunger rod in the reservoir/syringe. In addition, the assembly process of a disposable reservoir that includes a plunger rod is relatively complicated and lengthy due to the multiple steps involved in the assembly process. Furthermore, inseparably affixing a plunger rod to disposable reservoirs prevents storing or distributing, or otherwise treating such disposable reservoirs as ‘pre-filled’ devices. Therefore, conventional disposable reservoirs are typically filled up from a vial only a short time before they are used.

### SUMMARY OF THE INVENTION

A pump device for delivering medicament includes a disposable reservoir (e.g., a disposable syringe) and a reusable part that is detachably couplable to the disposable reservoir. The disposable reservoir includes, among other things, a plunger head (piston) that is bidirectionally moveable in the disposable reservoir between, for example, a proximal end of the disposable reservoir and a distal end of the disposable reservoir. The reusable part includes, among other things, a plunger rod (a leadscrew). The plunger head and the plunger rod are designed such that when the disposable reservoir and the reusable part are coupled to one another, the plunger rod can be releasably engaged with the plunger head as the plunger rod moves linearly, bi-directionally (back and forth), in the disposable reservoir in unison with the plunger head.

The plunger rod and the plunger head jointly include a quick connecting-release camming mechanism that enables the plunger rod to be quickly engaged with, and quickly released from, the plunger head. The camming mechanism includes a rotatable cylindrical cam body that is axially affixed to a distal end of the plunger rod linearly moveable between a stow (home) position in the reusable part of the pump device and a fully extended position. The camming mechanism is camingly rotatable about the plunger rod. The cam body includes cam surfaces to cammingly co-act with stationary cam pins in the plunger head to rotate in the plunger head to, and from, an engagement angular position in which the cam body and the plunger head are mutually engaged, to a disengagement angular position in which the cam body and the plunger head are disengageable. Rotation of the cam body in the plunger head between the engagement angular position and the disengagement angular position is performed via a series of alternating push-pull angular positions for alternately pushing and pulling the plunger head in the disposable reservoir. Rotating the cam body in the plunger head from one angular position to another is implemented by axially (linearly) moving the plunger rod in reciprocating manner, for example by an electric motor, to cause the cam pins of the plunger head to alternately and cammingly engage the cam surfaces of the cam body, to thereby cause the alternating pushing and pulling operation of the plunger rod and, consequently, of the plunger head, in the disposable reservoir.

### BRIEF DESCRIPTION OF THE DRAWINGS

Various exemplary embodiments and aspects are illustrated in the accompanying figures with the intent that these examples be not restrictive. It will be appreciated that for simplicity and clarity of the illustration, elements shown in the figures referenced below are not necessarily drawn to scale. Also, where considered appropriate, reference numerals that are repeated among figures indicate like, corresponding or analogous elements. Of the accompanying figures:

FIGS. 1A-1C show a pump device according to an example embodiment;

FIGS. 2A-2C show the plunger rod of FIG. 1 restrained against rotational movement by a rotation restrainer;

FIG. 3 shows a leadscrew type plunger rod according to an example embodiment;

FIG. 4 shows a plunger head according to an example embodiment;

FIGS. 5A-5B show a typical cylindrical cam body according to an example embodiment;

FIG. 6 shows the cylindrical cam body of FIGS. 2C and 5A-5B assembled on the plunger rod of FIG. 3, and before insertion into the plunger head of FIG. 4;

FIG. 7 shows a zig-zag channel in a cylindrical cam body, according to an example embodiment;

FIG. 8 is a spread-out view schematically illustrating example pulling members of a cylindrical cam body according to an example embodiment;

FIGS. 9A-9L are diagrammatic developed views illustrating step-by-step the manner in which cam surfaces of the cylindrical cam body of FIGS. 5A-5D are engaged alternately by the stationary cam pins of FIG. 4 by axially moving the plunger rod of FIG. 3 in reciprocating manner by the electric motor of FIG. 2C to effect an alternating pushing and pulling operation of the plunger rod and, consequently, of the plunger head; and FIGS. 10A-10I demonstrate typical operation of a four-stroke cycle pump device according to an example embodiment.

### DETAILED DESCRIPTION OF THE INVENTION

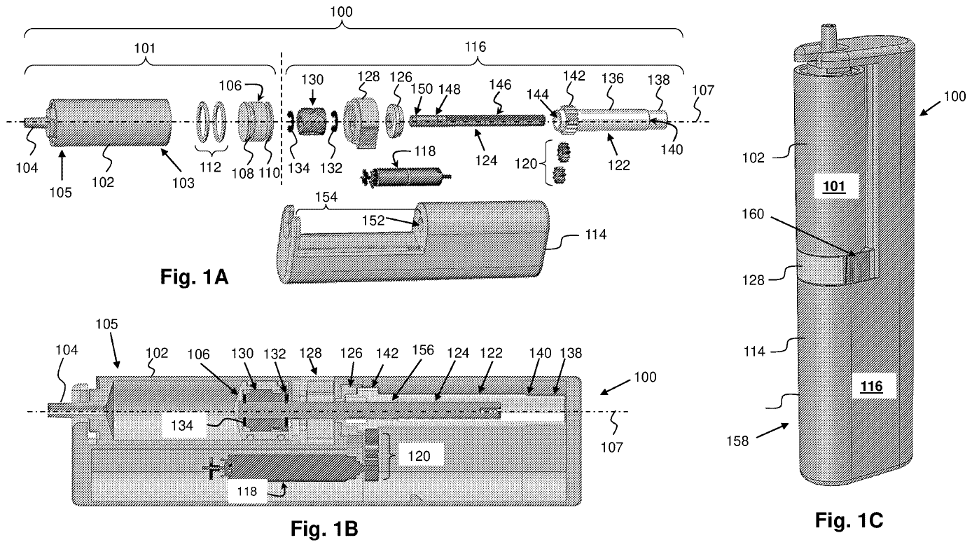

The description that follows provides various details of example embodiments. However, this description is not intended to limit the scope of the claims but instead to explain various principles of the invention and exemplary manners of practicing it. FIG. 1A, FIG. 1B and FIG. 1C respectively show an exploded view of a pump device (100), a cross-sectional view of pump device 100, and pump device 100 after assembly, according to an example embodiment. Pump device 100 includes a disposable medicament reservoir (for example in the form of a disposable syringe 101), and a reusable part 116. Syringe 101 includes a syringe housing 102, a syringe inlet/outlet port 104 through which syringe housing 102 can be filled with, and emptied from, liquid medicament, plunger head 106 with two annular grooves (108,110), and two O-rings 112 for two annular grooves 108 and 110. Plunger head 106 is bi-directionally moveable in syringe housing 102 between a first axial (proximal) position 103 on longitudinal axis 107 of syringe 101 and a second axial (distal) position 105 on longitudinal axis 107.

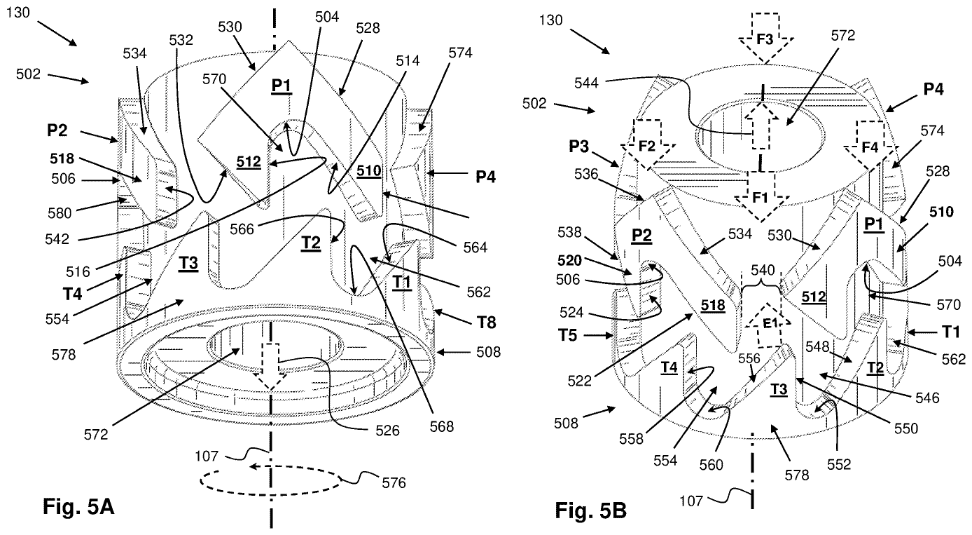

Reusable part 116 includes an electric motor 118, a gear train 120, a gear-driven shaft 122, a plunger rod 124 (a leadscrew), a plunger rod rotation restrainer 126, a syringe adapter 128 with a hand grip surface (160, FIG. 1C), a cylindrical cam body 130 and two flat retaining E-clip rings 132 and 134 (‘snap’ rings). Cylindrical cam body 130 includes a concentric through bore through which plunger rod 124 is inserted during the assembly process of the pump device. (The concentric bore in plunger rod 124 is shown in FIGS. 5A-5B, at 572.) Gear-driven shaft 122 includes an oblong hollow cylindrical body 136 with a proximal portion 138 and a distal end that includes a gear wheel 142. Gear wheel 142 is operatively coupled to, and drivable by, gear train 120. Proximal portion 138 of cylindrical body 136 has a reduced diameter with respect to the diameter of the main body of gear-driven shaft 122, to thus form a circumferential ledge 140 at proximal end 138 of gear-driven shaft 122. Circumferential ledge 140 is used to retain gear-driven shaft 122 in pump housing 114, as shown in FIG. 1B. Gear-driven shaft 122 includes a nut portion (the nut portion is not shown in FIG. 1A, but it is shown in FIG. 1B, at 156) and a bore, or sleeve, whose opening is shown at 144. The bore, or sleeve, may be a through bore, or through sleeve, meaning that they can have a second opening opposite opening 144.

Plunger rod 124 includes two threaded surfaces, one of which is shown at 146. (Two threaded surfaces 146 are positioned opposite one another, so the other threaded surface is not shown in FIG. 1A.) Plunger rod 124 also includes two circumferential grooves 148 and 150 that are recessed into the cylindrical body of plunger rod 124 to respectively receive retaining E-clip rings 132 and 134.

Reusable part 116 also includes a housing 114. Housing 114 includes a through hole 152 to enable electric motor 118 (in conjunction with gear train 120 and gear wheel 142) to bi-directionally move plunger rod 124 axially (linearly); namely, to extend plunger rod 124 out of housing 114, through hole 152, and to retract plunger rod 124 fully backwards to a stow position inside housing 114. Through hole 152 abuts bore/sleeve opening 144. Space 154 is for releasably and securely attaching syringe 101 and syringe adapter 128 to housing 114.

FIG. 1B, which shows pump device 100 after assembly and cylindrical cam body 130 axially fixed on a distal end of plunger rod 124 by (between) retaining E-clip rings 132 and 134. This means that cylindrical cam body 130 cannot move axially (along axis 107), along the length of plunger rod 124. Cylindrical cam body 130 is, however, freely rotatable about plunger rod 124. The way cylindrical cam body 130 is rotated about plunger rod 124 is described further below. FIG. 1B also shows plunger rod (leadscrew) 124 partially residing in the bore/sleeve in gear-driven shaft 122, with its two threaded surfaces 146 threadedly coupled to nut portion 156 of gear-driven shaft 122.

FIG. 1B also shows reusable part 116 and disposable syringe 101 releasably coupled to one another, for example by using a magnet (i.e., using magnetic attraction force), a snap-fit connector, a bayonet connector, etc. For example, a permanent magnet may be fixedly embedded in reusable part 116 or in disposable syringe 101, and a metal plate magnetically attractable to the magnet may be fixedly embedded in the other part (e.g., in disposable syringe 101, or in reusable part 116), such that when reusable part 116 and disposable syringe 101 are brought into proximity with one another, the permanent magnet would magnetically attract the metal plate. Cylindrical cam body 130 is shown in FIG. 1B residing in plunger head 106.

Referring to FIG. 1B, when motor 118 is activated, it rotates gear train 120, which, in turn, rotates gear wheel 142, hence the entire gear-driven shaft (122), about longitudinal axis 107 of plunger head 106, cylindrical cam body 130, gear-driven shaft 122 and plunger rod 124. Since nut portion 156 is part of or formed into cylindrical body 136 of gear-driven shaft 122, nut portion 156 and gear-driven shaft 122 are rotatable as one body/unit about rotational axis 107. Since gear-driven shaft 122 (hence nut portion 156) is axially restrained in reusable part 116 and plunger rod 124 is rotationally restrained by plunger rod rotation restrainer 126, rotation of nut portion 156 causes plunger rod 124 to move axially (linearly), along longitudinal axis 107. The direction at which plunger rod 124 moves along axis 107 depends on the direction of rotation (clockwise or anti-clockwise) of gear wheel 142, which, in turn, depends on the direction of rotation of electric motor 118; namely, changing the direction of rotation of electric motor 118 changes the direction of rotation of gear wheel 142, hence the direction of rotation of nut portion 156, hence the linear direction at which plunger rod 124 moves.

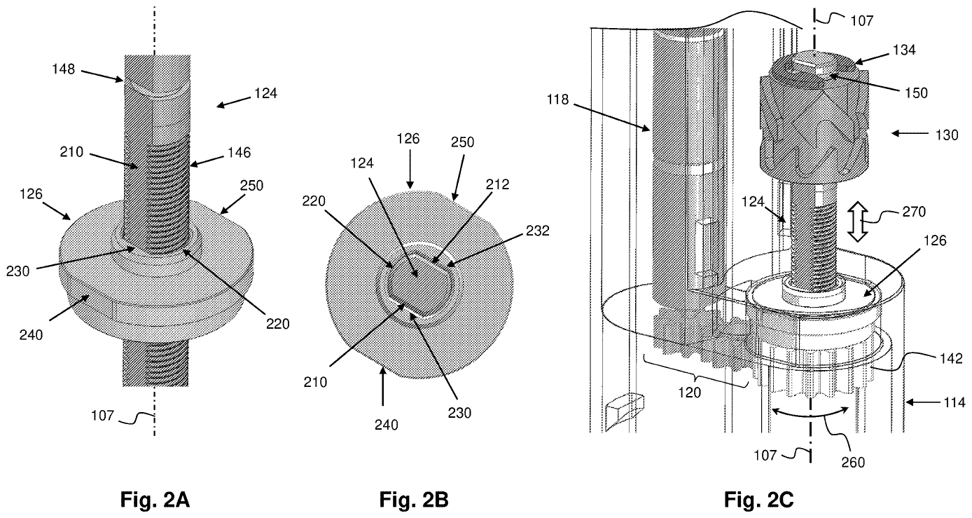

FIGS. 2A-2C show plunger rod 124 restrained by plunger rod restrainer 126 against rotational movement while enabling plunger rod 124 to move, or slide, linearly along longitudinal axis 107 of plunger rod 124. Referring to FIG. 2A, plunger rod 124 includes two flat truncation surfaces, one of which is shown at 210. (The other flat truncation surface 212 of plunger rod 124 is opposite flat truncation surface 210 and is shown in FIG. 2B.) Plunger rod restrainer 126 is generally a rounded object including a concentric through bore 220, through which plunger rod 124 can move (e.g., by electric motor 118) linearly (axially) along longitudinal axis 107. To restrain plunger rod 124 against rotational movement in plunger rod restrainer 126, through bore 220 of plunger rod restrainer 126 includes two restraining flat surfaces (230,232) that respectively abut truncation surfaces 210 and 212 of plunger rod 124. (Restraining flat surface 230 is shown at FIG. 2A, and both restraining flat surfaces 230 and 232 are shown in FIG. 2B.) Plunger rod restrainer 126 also includes two peripheral flat truncation surfaces (240,250) to prevent plunger rod restrainer 126 from rotating in housing 114, for example as a result of the rotation of nut portion 156 of gear-driven shaft 122 during normal operation of the pump device. FIG. 2B shows a top view of the plunger rod 124 and plunger rod restrainer 126.

FIG. 2C shows cylindrical cam body 130 axially fixed on the distal end of plunger rod 124 by (and between) flat retaining E-clip rings 132 and 134 (E-clip ring 132 is not shown in FIG. 2C). E-clip ring 134 is shown in FIG. 2C installed in an annular groove 150. Using retaining E-clip rings 132 and 134, cylindrical cam body 130 cannot move axially (along axis 107) on and along the length of plunger rod 124. Cylindrical cam body 130 can, however, rotate about plunger rod 124, between E-clip rings 132 and 134, by being provided with cam surfaces, as shown in FIG. 2C, and as described further below, for example in connection with FIGS. 5A-5B.

FIG. 2C also shows plunger rod restrainer 126 fixedly mounted in housing 114 of reusable part 116 of pump device 100. Also shown in FIG. 2C is part of the power transmission system of pump 100 that linearly moves plunger rod 124 along longitudinal axis 107. The power transmission system may include, among other things, electric motor 118, gear train 120, gear wheel 142 and a control circuit or system that may control operation of the power transmission system. Electric motor 118 may be selectively controlled to bi-directionally rotate (260) gear-driven shaft 122 (FIGS. 1A-1B) in a clockwise direction or in an anti-clockwise direction, to selectively move plunger rod 124 in a forward direction or in a rearward direction (270). FIG. 2C also shows annular (circumferential) grooves 108 and 110.

FIG. 3 shows plunger rod 124 more clearly. Plunger rod 124 has a proximal end 310 and a distal portion 320. Plunger rod 124 includes two threaded surfaces 146 that are separated by two flat truncated surfaces 210 and 212. (Only flat truncated surface 210 is shown in FIG. 3). Distal end 320 of plunger rod 124 includes two, axially spaced apart, annular (circumferential) grooves 148 and 150 for respectively accommodating two flat retaining E-clip rings 132 and 134.

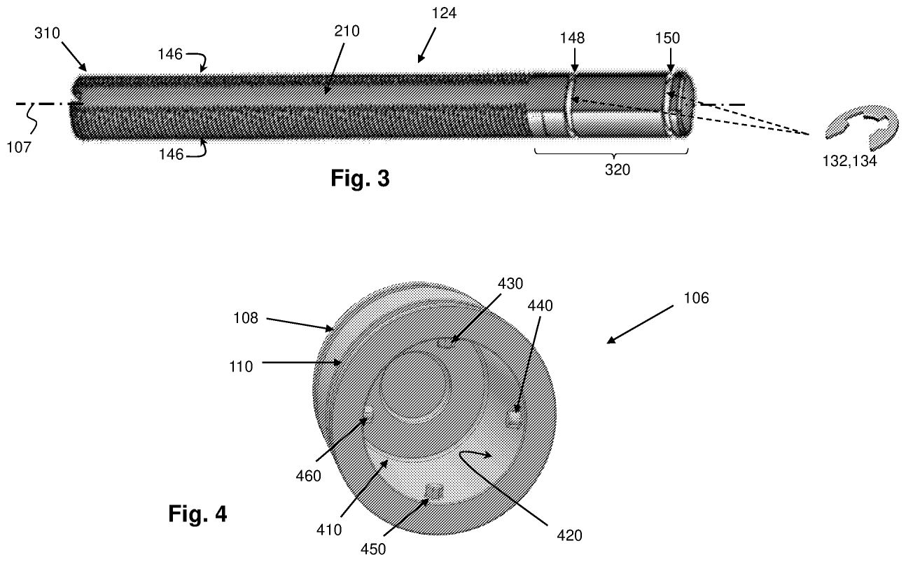

FIG. 4 shows cylindrical plunger head 106 according to an example embodiment. Plunger head 106 includes a one-sided open cylindrical chamber 410 that is concentrically formed in plunger head 106 by a cylindrical wall 420. Plunger head 106 also includes a plurality of cam pins that are equiangularly spaced around axis 107 of plunger head 106 and equiradially spaced from axis 107 and radially protrude from cylindrical wall 420 towards axis 107. (Longitudinal axis 107 is not shown in FIG. 4.) By way of example FIG. 4 shows four cam pins (e.g., cam pins 430, 440, 450 and 460). While plunger head 106 is bi-directionally linearly movable (e.g., by using electric motor 118) in syringe housing 102 along longitudinal axis 107, plunger head 106 is prevented from rotating in syringe housing 102 about axis 107 by a friction between the two O-rings (112), which are usually made of rubber, and the interior cylindrical wall of syringe housing 102. Cam pins 430, 440, 450 and 460 cammingly co-act with cam surfaces of cylindrical cam body 130 to rotate cylindrical cam body 130 in plunger head 106, about longitudinal axis 107, to various operational angular positions in plunger bead 106 The way this is done is described herein, for example in connection with FIGS. 5A-5B, 6, 7 and 9A-9L. Cylindrical cam body 130 may include a plurality of spaced cam surfaces that are located on the periphery of cylindrical cam body 130.

### Pulling Members: “U”—Shaped Pulling Recesses and Cam Surfaces

A cylindrical cam body may include a plurality of pulling members that are positioned on a distal end of the cylindrical cam body, and equiangularly spaced around axis 107 of cylindrical cam body 130 and equiradially spaced from axis 107 throughout their entire extents. Each pulling member includes a U-shaped pulling recess. The number of pulling members is identical to the number of cam pins. Each U-shaped pulling recess is configured to receive, and to pulingly engage with, a respective one of the cam pins to pull this pin cam. (All the cam pins are identical in size and shape, and all the U-shaped pulling recesses are identical in size and shape, because, during reciprocal axial movement of plunger rod 124, any cam pin may engage any U-shaped pulling recess, and any U-shaped pulling recess may engage any cam pin.) The U-shaped pulling recesses are used collectively to axially pull the cam pins, hence the plunger head 106, in syringe housing 102 when plunger rod 124 is moved backwards (rearwardly) in syringe housing 102, for example by electric motor 118.

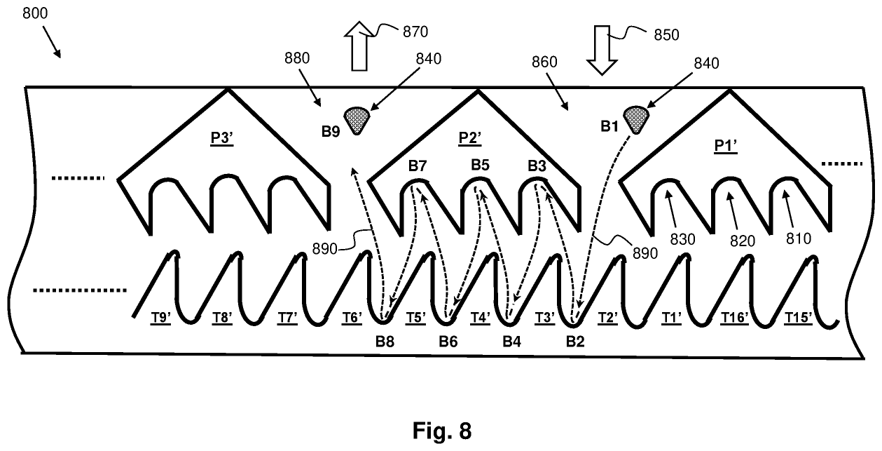

FIGS. 5A-5B show an example cylindrical cam body (130) that includes four example pulling members, which are denoted as P1, P2, P3 and P4, and each of these pulling members includes one pulling recess. However, a cylindrical cam body may include any mechanically useful number of pulling members, for example less than four pulling members (for example three pulling members), or more than four pulling members (for example five pulling members). In addition, a pulling member may include more than one pulling recess, for example two pulling recesses, three pulling recesses, etc. (Example pulling members including three pulling recesses are shown in FIG. 8.) The number of pulling members that a cylindrical cam body includes and the number of pulling recesses each pulling member may include depend, among other things, on mechanical aspects (e.g., overall size, obliqueness of the cam surfaces, diameter(s), shape, friction, materials used, etc.) of, for example, the cylindrical cam body, plunger head, cam pins and syringe. Any number of pulling members and pulling recesses may be used, subjected to mechanical feasibility.

Referring to FIGS. 5A-5B, pulling members P1, P2, P3 and P4 are identical in size, shape and spatial orientation (for example relative to longitudinal axis 107 of cylindrical cam body 130). Pulling members P1, P2, P3 and P4 include (provide) cam surfaces that are configured to operatively co-act with stationary cam pins 430, 440, 450 and 460 to rotate cylindrical cam body 130 in (relative to) cylindrical chamber 410 of plunger head 106 to a functionally desirable angular position upon reciprocating axial movement of plunger rod 124, hence of cylindrical cam body 130 in plunger head 106.

Each pulling member of pulling members P1, P2, P3 and P4 also includes a pulling recess. The pulling recesses are respectively pulingly engageable with cam pins 430, 440, 450 and 460 when the pulling recesses respectively abut the cam pins, and plunger rod 124 linearly (longitudinally, axially) pulls cylindrical cam body 130 in direction 526. Pulling member P1 includes an example pulling recess 504, and pulling member P2 includes an example pulling recess 506. (The pulling recesses of pulling members P3 and P4 are not shown in FIG. 5A-5B, but as explained herein, they are identical to pulling members P1 and P2 in terms of shape, size, spatial orientation, and function.) Each U-shaped pulling recess generally opens towards proximal end 508 of cylindrical cam body 130 at an acute angle with respect to a line parallel to longitudinal axis 107 of cylindrical cam body 130. (All U-shaped pulling recesses taper in parallel to axis line 107, towards distal end 502 of cylindrical cam body 130.)

Each pulling member Pi includes a first prong and a second prong, and the “U”-shaped pulling recess associated with pulling member Pi is formed by, and between, the first prong and the second prong. For example, pulling member P1 (FIGS. 5A-5B) includes a first prong (shown at 510) and a second prong (shown at 512). Prong 510 includes a cam surface 514 and prong 512 includes a stop surface 516. The U-shaped pulling recess in (associated with) pulling member P1 is formed by cam surface 514 and stop surface 516 of pulling member P1, and by a bottom surface (e.g., an arcuated surface, as shown at 504), that interconnects ('bridges') cam surface 514 and stop surface 516.

Similarly, pulling member P2 includes a first prong 518, which is identical to prong 510 of pulling member P1 in terms of structure, geometry, shape, orientation, and function, and a second prong 520 (FIG. 5B) that is identical to prong 512 of pulling member P1 in terms of structure, geometry, shape, orientation, and function Prong 518 includes a cam surface (522) that is identical to cam surface 514 of prong 510, and prong 520 includes a stop surface (524) that is similar to stop surface 516 of prong 512. Similarly to the U-shaped pulling recess 504 that is formed by pulling member P1, the U-shaped pulling recess of (associated with) pulling member P2 is formed by cam surface 522 and stop surface 524 of pulling member P2, and by a bottom surface (e.g., an arcuated surface, as shown at 506), that interconnects (‘bridges’) cam surface 522 and stop surface 524. The U-shaped pulling recesses of all other pulling members are formed by similar prongs in the same way as shown and described in connection with pulling members P1 and P2.

The cam surfaces of the first prongs of the pulling members (for example cam surface 514 of first prong 510 of pulling member P1, cam surface 522 of first prong 518 of pulling member P2, and so on) respectively co-act with cam pins 430, 440, 450 and 460 to rotate cylindrical cam body 130 in cylindrical chamber 410 of plunger head 106. Cylindrical cam body 130 rotates in cylindrical chamber 410 until cam pins 430, 440, 450 and 460 are respectively pulingly engaged with the pulling recesses (pulling recesses 504, 506 and so on) of pulling member P1, P2, P3 and P4. When cam pins 430, 440, 450 and 460 are respectively pulingly engaged with the pulling recesses of pulling member P1, P2, P3 and P4, electric motor 118 may controllably retract (move rearwardly) plunger rod 124 in syringe housing 102 to pull cylindrical cam body 130, hence plunger head 106 in syringe housing 102, rearwardly (in direction 526), for example to fill up syringe housing 102 with medicament.

Each pulling member includes three additional cam surfaces (and, per the example described herein, a total of four cam surfaces). By way of example, pulling member PI also includes cam surfaces 528, 530 and 532, and pulling member P2 also includes cam surfaces 534, 536 and 538.

In reference to pulling member P1, each of cam surfaces 528 and 530 (jointly with the corresponding cam surfaces of the other pulling members) may co-act with one of cam pins 430, 440, 450 and 460 to rotate cylindrical cam body 130 in cylindrical chamber 410 to an angular position in which cylindrical cam body 130 can receive all cam pins 430, 440, 450 and 460 and co-act with them to rotate cylindrical cam body 130 to various operational angular positions. The various operational angular positions of cylindrical cam body 130 relative to the cam pins, hence to the plunger head, correspond to different phases of the reciprocating movement of plunger rod 124 including engagement of plunger rod 124 with plunger head 106 (by means of cylindrical cam body 130), alternately pushing and pulling plunger head 106 in syringe housing 102 (by means of cylindrical cam body 130), and, finally, releasing (disengaging) plunger rod 124 from plunger head 106 by releasing plunger rod 124 from cylindrical cam body 130. The various operational angular positions corresponding to the different activations of plunger rod 124 are described herein, for example further below.

In reference to pulling member P2, cam surfaces 534 and 536 function in the same way as cam surfaces 528 and 530 of pulling member P1, though through coaction with a different one of cam pins 430, 440, 450 and 460. All other pulling members likewise include two cam surfaces of the same type as, for example, cam surfaces 528 and 530. All cam surfaces and stop surfaces of cylindrical cam body 130 are equiangularly spaced around axis 107 of cylindrical cam body 130 and equiradially spaced from axis 107 throughout their entire extents.

FIGS. 5A-5B show an example cylindrical cam body 130 that includes four example pulling members, which are denoted as P1, P2, P3 and P4, and each of these pulling members include one pulling recess. By way of example, each of pulling members P1, P2, P3 and P4 includes one pulling recess.

### Pulling Members—Through Channels: Entrance Funnels and Exit Funnels

Each two adjacent pulling members (Pi, Pitt) jointly form (define) therebetween a through channel, via which a respective one of cam pins 430, 440, 450 and 460 may enter cylindrical cam body 130, or exit cylindrical cam body 130. (During reciprocating axial movement of plunger rod 124 a cam pin does not enter and exit cylindrical cam body 130 via the same through channel. That is, if a cam pin, for example cam pin 430, enters cylindrical cam body 130 via a specific through channel, the cam pin exits cylindrical cam body 130 only via a different, subsequent, through channel.)

A through channel includes an entrance funnel and an exit funnel. The number of the through channels is identical to the number of the cam pins. The through channels and the cam pins are configured such that each particular cam pin abuts an entrance funnel of a particular one of the through channels for insertion into (i.e., engaging) cylindrical cam body 130 when plunger rod 124 moves cylindrical cam body 130 forward (544) into chamber 410 of plunger head 106, and an exit funnel of a different through channel for extraction of (withdrawing) from cylindrical cam body 130 when plunger rod 124 moves cylindrical cam body 130 rearwardly (526), and away, from chamber 410 of plunger head 106. (The entrance funnels axially precede the exit funnels into cylindrical cam body 130.)

An entrance funnel Fi is formed by an axially anterior pair of conjugated cam surfaces that includes an anterior cam surface of a particular pulling member Pa and an anterior cam surface of a pulling member Piti that is adjacent to the particular pulling member (Pi). An exit funnel Ei is formed by an axially posterior pair of conjugated surfaces that includes a posterior cam surface of a particular pulling member Pi and a posterior surface of an adjacent pulling member Pᵢ₊₁. By way of example (referring to FIG. 5B), entrance funnel F1 is formed by an axially anterior pair of conjugated cam surfaces that includes anterior cam surface 530 of pulling member P1 and anterior cam surface 534 of pulling member P2 that is adjacent to pulling member P1. Similarly (referring to FIGS. 5A-5B), entrance funnel F4 is formed by an axially anterior pair of conjugated cam surfaces that includes anterior cam surface 574 of pulling member P4 and anterior cam surface 528 of pulling member PI that is adjacent to pulling member P4.

By way of example (referring again to FIG. 5B), exit funnel El is formed by an axially posterior pair of conjugated surfaces that includes posterior cam surface 532 of pulling member P1 and posterior stop surface 542 of pulling member P2 that is adjacent to pulling member P1. (‘Anterior’ and ‘posterior’ locations, as used herein, are axial locations relative to the cylindrical cam body 130. For example, being “axially anterior”, cam surface 530 of pulling member P1 and cam surface 534 of pulling member P2 precede cam surface 532 of pulling member P1 and surface 542 of pulling member P2 into cylindrical cam body 130.)

Each entrance funnel is configured to rotatively receive and guide a respective one of the cam pins into an engagement angular position upon linear insertion of cylindrical cam body 130 into chamber 410 of plunger head 106 by plunger rod 124. Linear (axial, 107) and irrotational movement of cylindrical cam body 130 forward, in direction 544, into chamber 410 of plunger head 106 causes the corresponding cam surfaces of cylindrical cam body 130 to co-act with cam pins 430, 440, 450 and 460 to rotate cylindrical cam body 130 about longitudinal axis 107 to an angular position in chamber 410 in which the cam pins are respectively engaged by the pushing recesses, in which position plunger rod 124 can push cam pins 430, 440, 450 and 460 in unison, hence plunger head 106, in syringe housing 102.

Conversely, axial (107) and irrotational movement of cylindrical cam body 130 backwards (rearwardly), in direction 526, into or in chamber 410 of plunger head 106 causes the corresponding cam surfaces (e.g., other cam surfaces) of cylindrical cam body 130 to co-act with cam pins 430, 440, 450 and 460 to rotate cylindrical cam body 130 about longitudinal axis 107 to an angular position in chamber 410 in which the cam pins are respectively engaged by the pulling recesses, and plunger rod 124 can pull cam pins 430, 440, 450 and 460 in unison, hence plunger bead 106 in syringe housing 102.

Referring to FIGS. 5A-5B, entrance funnel F1 is configured to rotatively receive and guide a respective one of cam pins 430, 440, 450 and 460 into an engagement angular position in cylindrical cam body 130 upon linear insertion of cylindrical cam body 130 into chamber 410 by plunger rod 124. (Linear movement of cylindrical cam body 130 in direction 544 against the cam pins causes cylindrical cam body 130 to rotate about axis 107.) FIGS. 5A-5B show an example cylindrical cam body (130) with four example pushing members. Accordingly, there are four through channels in total, and each through channel includes an opening similar to opening 540. FIG. 5B shows the other three entrance funnels, namely, F2 (which is jointly defined by, and in-between, pulling member P2 and pulling member P3), F3 (which is jointly defined by, and in-between, pulling member P3 and pulling member P4) and F4 (which is jointly defined by, and in-between, pulling member P4 and pulling member P1).

Referring to FIG. 5B, exit funnel El is configured such that cylindrical cam body 130 rotatively receive and guide a respective one of cam pins 430, 440, 450 and 460 out of a disengagement angular position in cylindrical cam body 130 upon linear retraction of cam body 130 from chamber 410 of plunger head 106. (Linear movement of cylindrical cam body 130 away from the cam pins causes cylindrical cam body 130 to rotate about axis 107 until the cam pins can be respectively released from cylindrical cam body 130 via the exit funnels.)

Each entrance funnel (for example entrance funnel F1) has a large opening at distal end 502 of cylindrical cam body 130, and it tapers (narrows down) from distal end 502 towards the proximal end 508 of cylindrical cam body 130. Each exit funnel (e.g., exit funnel E1) has a large opening that opens towards proximal end 508 of cylindrical cam body 130, and it tapers (narrows down) towards distal end 502 of cylindrical cam body 130. Accordingly, the entrance funnel (F1, FIG. 5B) and the exit funnel (E1, FIG. 5B) of each through channel are lengthwise positioned ‘back-to-back’ such that the narrow opening of the entrance funnel and the narrow opening of the exit funnel coincide. Referring to FIG. 5B, entrance funnel F1 and exit funnel E1 are lengthwise positioned back-to-back such that the narrow opening of the entrance funnel and the narrow opening of the exit funnel coincide as opening 540.

### Teeth—Cam Surfaces and Pushing Recesses

Cylindrical cam body 130 includes a number ‘n’ of teeth that are circumferentially and equiangularly spaced around axis 107, and equiradially spaced from axis 107 throughout their entire extent and arranged on the periphery of cylindrical cam body 130 to provide ‘n’ cam surfaces and ‘n’ stop surfaces on a proximal end of the cylindrical cam body. The ‘n’ cam surfaces and ‘n’ stop surfaces provided by the ‘n’ teeth jointly form ‘n’ “U”-shaped pushing recesses for enabling plunger rod 124 to push plunger head 106, via the cam pins, for example to expel medicament that is contained in syringe housing 102. (Expelling medicament from the syringe housing may be performed, for example, for priming an infusion tubing set and/or to deliver medicament to a patient, e.g., subcutaneously.)

By way of example, cylindrical cam body 130 includes eight (n=8) circumferential teeth, which are orderly denoted as T1, T2, T3, T4, TS, T6, T7 and T8. Teeth T1-T8 are circumferentially, equiangularly and equiradially spaced apart on proximal end 508 of cylindrical cam body 130, around longitudinal axis 107 of cylindrical cam body 130 (FIG. 5A shows teeth T1, T2, T3, T4 and TS. FIG. 5B shows teeth T1, T2, T3, T4 and T5.) Circumferential teeth T1, T2, T3, T4, TS, T6, T7 and T8 jointly form a serrated crown 578 in proximal end 508 of cylindrical cam body 130, and each tooth of serrated crown 578 is a right-angled triangle that includes a cam surface and a stop surface.

Teeth T1, T2, T3, T4, TS, T6, T7 and T8 form, therebetween, eight structurally identical “U”-shaped pushing recesses. Each particular “U”-shaped pushing recess is formed by the cam surface of a particular tooth (Ti) and the stop surface of a subsequent tooth (Ti+1). and by a bottom surface that interconnects the cam surface of the particular tooth (Ti) and the stop surface of the subsequent (consecutive) tooth (Ti+1) and forms therewith one contiguous surface. By way of example (referring to FIG. 5B), “U”-shaped pushing recess 546 is formed between teeth T2 and T3, that is, by cam surface 548 of tooth T2 and stop surface 550 of tooth T3, and by a bottom surface (552) that interconnects cam surface 548 and stop surface 552. (Cam surface 548, stop surface 550 and bottom surface 552 form one contiguous surface.)

Similarly, U-shaped pushing recess 554 is formed between teeth T3 and T4, that is, by cam surface 556 of tooth T3 and stop surface 558 of tooth T4, and by a bottom surface (560) that interconnects cam surface 556 and stop surface 558 and forms therewith one contiguous surface. (Cam surface 556, stop surface 558 and bottom surface 560 form one contiguous surface.) Similarly, U-shaped pushing recess 562 is formed between teeth T1 and T2, that is, by cam surface 564 (FIG. 5A) of tooth T1 and stop surface 566 (FIG. 5A) of tooth T2, and by a bottom surface (568) that interconnects cam surface 564 and stop surface 566 and forms therewith one contiguous surface. The rest of the U-shaped pushing recesses are formed in a similar way between teeth T4 and T5, between teeth T5 and T6, between teeth T6 and T7, between teeth T7 and T8, and between teeth T8 and T1. (Cam surface 564, stop surface 566 and bottom surface 568 form one contiguous surface.)

The cam surfaces of the teeth (for example cam surface 564 of tooth TI, cam surface 548 of tooth T2, cam surface 556 of tooth T3, and so on) respectively co-act with cam pins 430, 440, 450 and 460 to rotate cylindrical cam body 130 in cylindrical chamber 410 of plunger head 106. Cylindrical cam body 130 rotates in cylindrical chamber 410 until cam pins 430, 440, 450 and 460 are respectively engaged with the pushing recesses (pushing recesses 562, 546, 554, and so on). When cam pins 430, 440, 450 and 460 are respectively engaged with the pushing recesses, electric motor 118 may controllably push (move forward) plunger rod 124 in syringe housing 102 to push cylindrical cam body 130, hence plunger head 106 in syringe housing 102, forward (in direction 544), for example to expel medicament from syringe housing 102, for example to prime an infusion tubing set and/or to deliver medicament to a patient.

The cam surfaces of cylindrical cam body 130 may follow, for example, a 30°helix around cylindrical cam body 130, and they are configured to impart a unidirectional rotational movement to cylindrical cam body 130 when cylindrical cam body 130 is reciprocally axially moved (by plunger rod 124) against the rotationally fixed cam pins of plunger head 106. (Plunger rod 124 is repeatedly moved axially forward and rearwardly to align alternate cam surfaces with the stationary cam pins to maintain the cam body 130 alternately in plunger head pushing position and plunger head pulling position.)

### Unidirectional Rotation of the Cylindrical Cam Body

In general, the direction of the cam surfaces of the pulling members and teeth of cylindrical cam body 130 is such that reciprocally moving cylindrical cam body 130 in chamber 410 of plunger head 106 by plunger rod 124 along rotational axis 107 imparts unidirectional rotational movement (576, FIG. 5A), as well as longitudinal reciprocating movement, to cylindrical cam body 130 in chamber 410. (The unidirectional rotation direction, in this example rotation direction 576, is referred to herein as the “main rotation direction”) This notion, however, has one conditional exception that is related, among other things, to the entry funnels, as described hereinafter.

Prior to engagement of plunger rod 124 with plunger head 106 via cylindrical cam body 130 and the cam pins (i.e., before cylindrical cam body 130 is pushed by plunger rod 124 into chamber 410 of plunger head 106), cylindrical cam body 130 is free to rotate on plunger rod 124 between two retaining E-clip rings 132 and 134. This means that the initial angular orientation of entry funnels F1, F2, F3 and F4 of cylindrical cam body 130 relative to the angularly stationary cam pins of chamber 410 is random. In addition, the angular orientation of chamber 410, hence of the cam pins, relative to the syringe housing may vary (e.g., during assembly of the syringe) from one syringe to another, which adds another factor to the “entry funnel”-to-“cam pins” alignment randomicity.

So, the anterior cam surfaces of the entry funnels that can potentially rotate cylindrical cam body 130 (‘potentially’—if these cam surfaces co-act with the cam pins) in the same main rotation direction 576 as the other cam surfaces of cylindrical cam body 130 are not necessarily angularly aligned with the cam pins at the time of engagement between cylindrical cam body 130 and the cam pins. An anterior cam surface of an entry funnel that, when cammingly co-acted by a cam pin, maintains the main rotation direction 576 is referred to herein as a ‘primary cam surface’. (Entry funnel cam surfaces 530, 536 and 574 are example primary cam surfaces.) However, it may occur that the funnels' anterior cam surfaces that can potentially rotate cylindrical cam body 130 in main rotation direction 576 are angularly misaligned with the cam pins; that is, they may be angularly offset relative to the cam pins at the time of engagement between cylindrical cam body 130 and the cam pins. So, in case of angular misalignment between the primary cam surfaces and the cam pins, the cam surfaces that may be aligned with the cam pins are the other anterior cam surfaces of the entry funnels, which are referred to herein as ‘auxiliary cam surfaces’. (Entry funnel cam surfaces 528 and 534 are example auxiliary cam surfaces.) In this case, co-action between the auxiliary cam surfaces and the cam pins would cause cylindrical cam body 130 to rotate in direction that is opposite to main rotation direction 576. There is a third option, though, which is that the cam pins are aligned with the openings 540 in the through channels, in which case the cam pins will respectively reach the cam surfaces of the teeth abutting the through channels without causing cylindrical cam body 130 to rotate in any direction.

The U-shaped pushing recesses are configured to axially push the cam pins (e.g., cam pins 330, 340, 350 and 360) in unison, hence plunger head 106, in the forward direction (in direction 544) in syringe housing 102 when plunger rod 124 is moved forward, for example by electric motor 118, to move cylindrical cam body 130 in direction 544, for example to expel medicament from syringe housing 102.

In an example embodiment, the number of teeth (‘n’) may be twice the number of pulling members, ‘k’ (e.g., k=n/2). Continuing this example, the number of pulling members is four (k=4), as shown, for example, in FIGS. 5A-5B, which show example pulling members P1, P2, P3 and P4. Each U-shaped pushing recess generally opens towards distal end 502 of cylindrical cam body 130 at an acute angle with respect to a line parallel to a longitudinal axis of cam body 130. (All U-shaped pushing recesses taper in parallel to axis line 107, towards proximal end 508 of cylindrical cam body 130.)

The ‘n’ circumferential teeth and the ‘n/2’ pulling members are peripherally disposed on the cylindrical cam body in alternating relation such that every odd tooth T₂ᵢ₋₁ (e.g., tooth T1, T3, T5, and so on) structurally abuts (is located opposite) a through channel that is jointly formed by and between two adjacent pulling members Pᵢ and Pᵢ₊₁, and every even tooth T₂ᵢ (e.g., tooth T2, T4, T6, and so on) structurally abuts (is located opposite) a U-shaped pulling recess of a particular pulling member (Pitt). By way of example, teeth T1, T2, T3, T4, T5, T6, T7 and T8 and pulling members P1, P2, P3 and P4 are peripherally disposed on cylindrical cam body 130 in alternating relation such that
Tooth T1 abuts the through channel that is jointly formed by and between adjacent pulling members P1 and P4;
Tooth T2 abuts U-shaped pulling recess 570 of pulling member P1;
Tooth T3 abuts the through channel that is jointly formed by and between adjacent pulling members P1 and P2;
Tooth T4 abuts U-shaped pulling recess 580 (FIG. 5A) of pulling member P2, and so on.

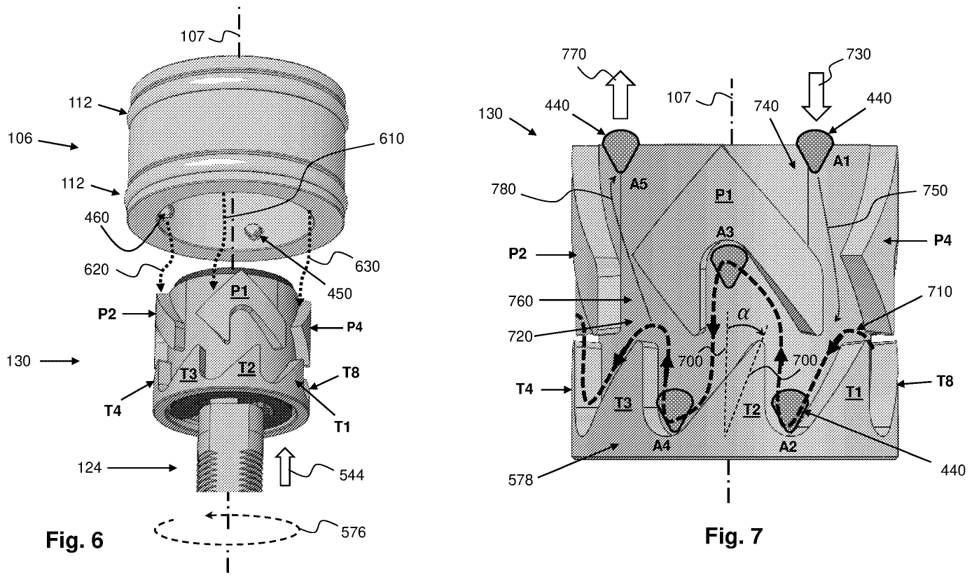

FIG. 6 show cylindrical cam body 130 and plunger rod 124 assembled together, and cylindrical cam body 130 is shown prior to insertion into plunger head 106 by plunger rod 124; namely
Cam pin 430 (not shown in FIG. 6), which is located radially opposite cam pin 450, abuts (610) the through channel formed by and between pulling members P1 and P2;
Cam pin 460 of plunger head 106 abuts (620) the through channel formed by and between pulling members P2 and P3 (pulling member P3 is not shown in FIG. 6);
Cam pin 450 of plunger head 106 abuts the through channel formed by and between pulling members P3 and P4 (pulling member P3 is not shown in FIG. 6), and
Cam pin 440 (not shown in FIG. 6), which is located radially opposite cam pin 460, abuts (630) the through channel formed by and between pulling members P4 and P1.

In the example orientation of cylindrical cam body 130 relative to plunger head 106 shown in FIG. 6, the cam surfaces of cylindrical cam body 130 that abut the cam pins are the rotation-keeping cam surfaces. Therefore, when plunger rod 124 is linearly (axially) moved, for example by electric motor 118, in the forward direction (544) along longitudinal axis 107, the anterior cam surfaces of cylindrical cam body 130 cammingly co-act with the cam pins of plunger head 106 to rotate cylindrical cam body 130 in direction 576.

The ‘n/2’ pulling members are axially distanced away from the ‘n’ teeth of the serrated crown and define, therewith, a guiding ‘zig-zag’ channel for traversal by the plurality of cam pins upon rotation of the cylindrical cam body in the plunger head from the engagement angular position, in which the cam pins respectively abut the through channels, to the disengagement. angular position (in which the cam pins respectively abut the through channels) via a series of alternating pushing-pulling angular positions upon reciprocating axial movement of the plunger rod. (The guiding zig-zag channel guides the cam pins in the cylindrical cam body from one recess to another, and, correspondingly, from one operating angular position of the cylindrical cam body to another.)

Referring to FIG. 7, pulling members P1, P2, P3 and P4 (pulling member P3 is not shown in FIG. 7) are axially distanced away from serrated crown 578 to define, therewith, a guiding ‘zig-zag’ channel, which is shown in FIG. 7 by dotted (dashed) line 710. (The five arrows on dotted line 710 denote the movement direction of a cam pin relative to zig-zag channel 710 when cylindrical cam body 130 is rotated in direction 576 and reciprocally moved in plunger head 106.)

Guiding zig-zag channel 710 encircles external surface 720 of cylindrical cam body 130, and each cam pin of plunger head 106 enters, or accesses, guiding zig-zag channel 710 via the opening (e.g., 540, FIG. 5B) in the corresponding through channel. (The opening of each through channel may be referred to as an access point of zig-zag channel 710) The width of guiding zig-zag channel 710 changes along the length of the channel, but its minimal value is set so as to enable traversal of the channel by the cam pins 430, 440, 450 and 460 when cylindrical cam body 130 is rotated in plunger head 106.

A rotation ‘cycle’ of cylindrical cam body 130 in plunger head 106 includes rotation of cylindrical cam body 130 from an engagement angular position, in which the cam pins of plunger head 106 respectively abut the entry funnels, to a disengagement angular position, in which the cam pins respectively abut the exit funnels such that each particular cam pins abuts an exit funnel of a through channel that is subsequent to the through channel including the entry funnel of the particular cam pin. Rotation of cylindrical cam body 130 from the engagement angular position to the disengagement angular position is done via a contiguous series of alternating pushing-pulling angular positions upon reciprocating axial movement of plunger rod 124, as described herein, for example, hereinafter.

For the sake of brevity, FIG. 7 shows traversal of zig-zag channel 710 by one example cam pin (e.g., cam pin 440). (The other cam pins of the plunger head enter zig-zag channel 710 at different access points and traverse a different portion of zig-zag channel 710.) When cylindrical cam body 130 is pushed forward (544) into plunger head 106 by plunger rod 124 (as shown in FIG. 6), cam pin 440 enters (730) cylindrical cam body 130 and accesses zig-zag channel 710 by entering entry funnel 740 and moving along entry through path 750. Cam pin 440 is stationary while cylindrical cam body 130 is rotated by cam pin 440 (and by the other cam pins of the plunger head) from one angular position to another as cylindrical cam body 130 reciprocally axially moves in plunger head 106 by reciprocating plunger rod 124.

As cam pin 440 enters entry funnel 740 (by pushing cylindrical cam body 130 against cam pin 440) via landmark A1, cam pin 440 either rotates cylindrical cam body 130 in direction 576 or in the opposite direction, depending on which anterior cam surface (rotation-keeping cam surface or counter-rotation cam surface) is co-acted by cam pin 440. If none of these cam surfaces is co-acted by cam pin 440, cam pin 440 will access zig-zag channel 710 following entry path 750 without rotating cylindrical cam body 130.

To simplify the description, cam pin 440 is shown in FIG. 7 at five different times and landmarks (which are denoted as A1, A2, A3, A4 and A5) along its path relative to cylindrical cam body 130, to illustrate the various angular positions that cylindrical cam body 130 is rotated to by, and relative to, stationary cam pin 440. Landmark Al corresponds to the engagement angular position of cylindrical cam body 130 (relative to cam pin 440) in which cylindrical cam body 130 is engageable with plunger head 106 by moving cylindrical cam body 130 in forward direction 544 against stationary plunger head 106. Landmarks A2 through A4 signify the contiguous series of alternating pushing-pulling angular positions of cylindrical cam body 130 relative to cam pin 440. Landmark AS corresponds to the disengagement angular position (relative to cam pin 440) in which cylindrical cam body 130 can be disengaged from plunger head 106 by moving cylindrical cam body 130 away from stationary plunger head 106.

Regarding landmarks A2 through A4, landmark A2 signifies a first U-shaped pushing recess corresponding to a first pushing angular position in which plunger rod 124 can push plunger head 106 forward, landmark A3 signifies a U-shaped pulling recess corresponding to a pulling angular position in which plunger rod 124 can pull plunger head 106 rearwardly, and landmark A4 signifies a second U-shaped pushing recess corresponding to a second pushing angular position in which plunger rod 124 can push plunger bead 106 forward again, hence the alternating nature′ of the pushing recess(es) and the pulling recess(es).

When cam pin 440 is at landmark A4 (after zig-zigging along zig-zag channel 710), cylindrical cam body 130 may be first pushed forward (544) by plunger rod 124 (for example to expel medicament from syringe housing 102) and then, pulled by plunger rod 124 in order to disengage cylindrical cam body 130 from cam pin 440. Disengaging cam pin 440 from cylindrical cam body 130 is done by pulling cylindrical cam body 130 rearwardly to cause cam pin 440 to leave zig-zag channel 710, enter exit funnel 760 and, in general, move out (770) of cylindrical cam body 130 along exit through path 780.

The example shown in FIG. 7 shows cylindrical cam body with a specific design where each of pulling members P1, P2, P3 and P4 includes one pulling recess. This design (Le., a pulling member includes one pulling recess) provides, for each cam pin, a series of three alternating pushing-pulling angular positions, and five angular positions altogether, including the engagement angular position (A1), the series of three alternating pushing-pulling angular positions (A2 through A4), and the disengagement angular position (A5). However, in other embodiments the cylindrical cam body may be designed to provide a different number of pulling angular positions and pulling angular positions, as described below in connection with FIG. 8. The zig-zag channel formed in the cylindrical cam body (e.g., zig-zag channel xxx, Fig. xx) guides the cam pins in the cylindrical cam body from one recess to another, and, correspondingly, from one operating angular position of the cylindrical cam body to another.)

FIG. 8 schematically illustrates a spread-out cylindrical cam body 800 according to another embodiment. Similarly to cylindrical cam body 130, cylindrical cam body 800 includes four pulling members, which are denoted as P1′, P2′, P3′ and P4′. However, unlike cylindrical cam body 130, each of pulling members P1′, P2′, P3′ and P4′ includes (provides) three pulling recesses. For example, pulling members PI′ includes pulling recesses 810, 820 and 830. Accordingly, cylindrical cam body 800 provides twelve pulling recesses in total. The four pulling members P1′, P2′, P3′ and P4′ define, therebetween, four through channels. Accordingly, cylindrical cam body 800 is also designed to co-act with a plunger head that is similar to plunger bead 106, which includes four cam pins. Cam body 800 also includes sixteen teeth, which are denoted as T1′, T2′, T3′, . . . , T16′, which define therebetween sixteen pushing recesses that may resemble teeth T1, T2, T3, . . . , T8 of cylindrical cam body 130.

As described in connection with cylindrical cam body 130, a cam pin (e.g., cam pin 440) enters cylindrical cam body 130 via a particular through channel, and leaves cam body 130 via a subsequent (consecutive) through channel. Similarly, cam pin 840 enters (850) cylindrical cam body 800 via through channel 860, and leaves (870) cylindrical cam body 800 via subsequent (consecutive) through channel 880.

Cam pin 840 is shown in FIG. 8 at nine different times and landmarks (which are denoted as B1, B2, B3, B4, B5, B6, B7, B8, and B9) along zig-zag path 890 relative to cylindrical cam body 800. The nine landmarks illustrate the various angular positions that cylindrical cam body 800 is successively rotated to by, and relative to, stationary cam pin 840. Landmark BI corresponds to the engagement angular position of cylindrical cam body 800 (relative to cam pin 840) in which cylindrical cam body 800 is engageable with the plunger head by moving cylindrical cam body 800 in forward direction against the stationary plunger head. Landmarks B2 through BS signify the contiguous series of alternating pushing-pulling angular positions of cylindrical cam body 800 relative to cam pin $40. Landmark B9 corresponds to the disengagement angular position (relative to cam pin 840) in which cylindrical cam body 800 can be disengaged from the plunger head by moving cylindrical cam body 800 away from the stationary plunger head.

Regarding landmarks B2 through B8, landmark B2 signifies a first U-shaped pushing recess corresponding to a first pushing angular position in which the plunger rod can push the plunger head forward. Landmark B3 signifies a U-shaped pulling recess corresponding to a first pulling angular position in which the plunger rod can pull the plunger head rearwardly. Landmark B4 signifies a second U-shaped pushing recess corresponding to a second pushing angular position in which the plunger rod can push the plunger head forward again. Landmark B5 signifies a U-shaped pulling recess corresponding to a second pulling angular position in which the plunger rod can pull the plunger head rearwardly. Landmark BG signifies a third U-shaped pushing recess corresponding to a third pushing angular position in which the plunger rod can push the plunger head forward again. B7 signifies a U-shaped pulling recess corresponding to a third pulling angular position in which the plunger rod can pull the plunger head rearwardly. Landmark BS signifies a fourth U-shaped pushing recess corresponding to a fourth pushing angular position in which the plunger rod can push the plunger head forward again, hence the alternating ‘nature’of the pushing recess(es) and the pulling recess(es).

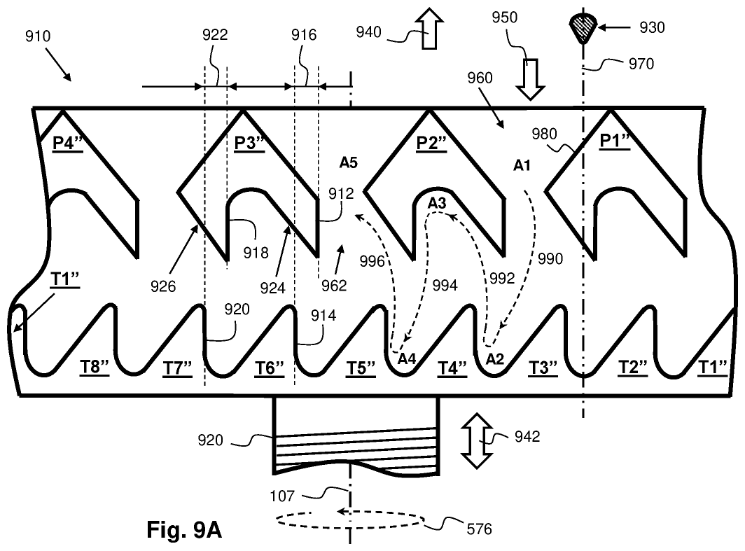

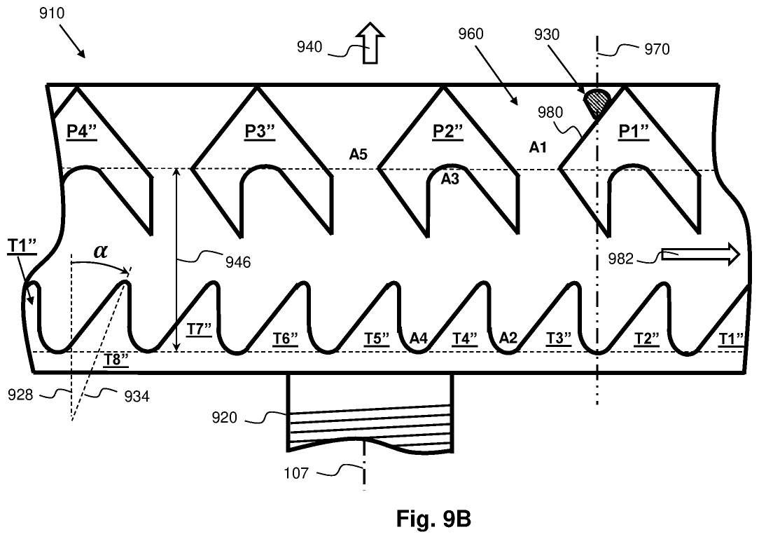

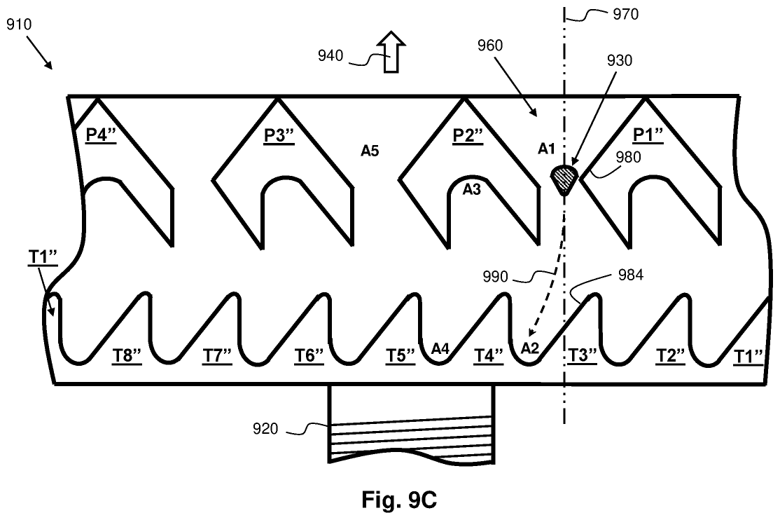

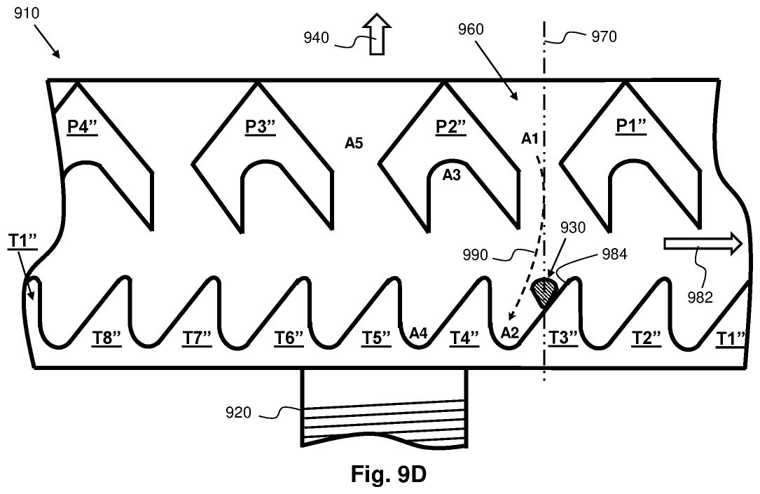

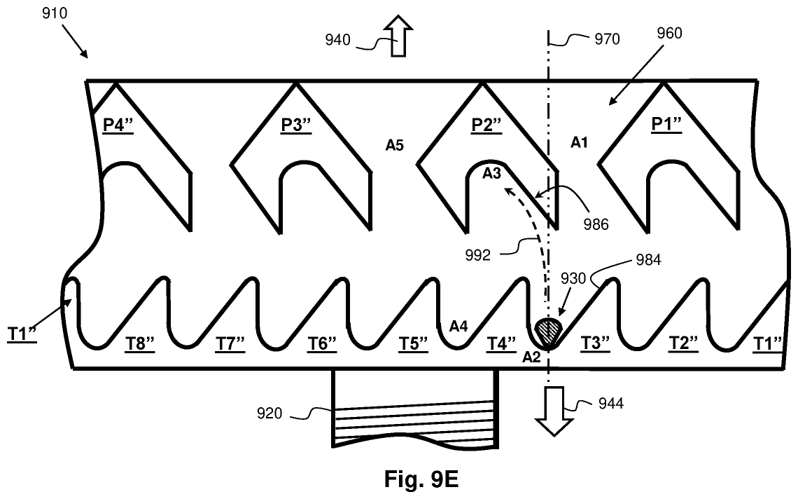

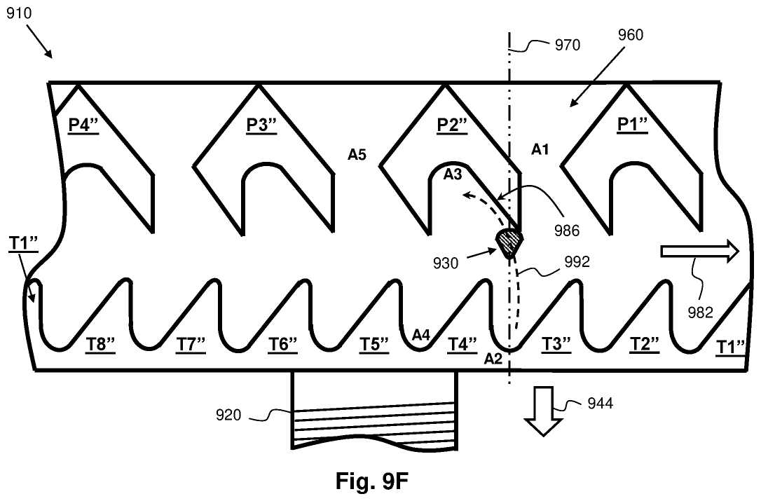

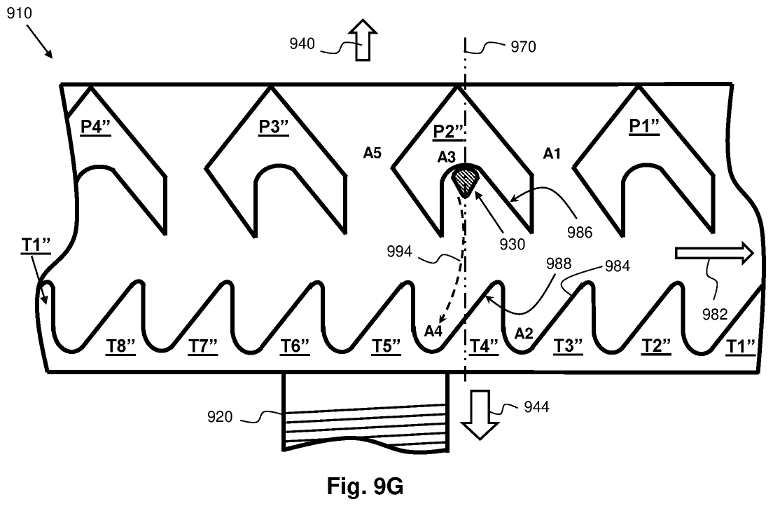

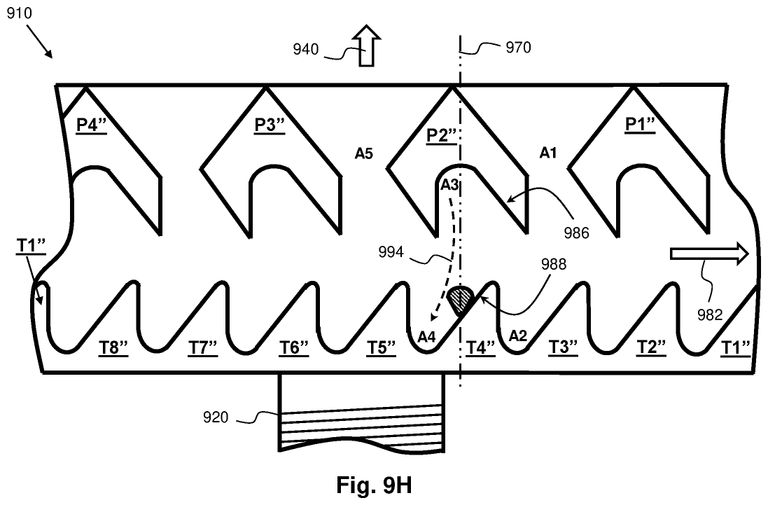

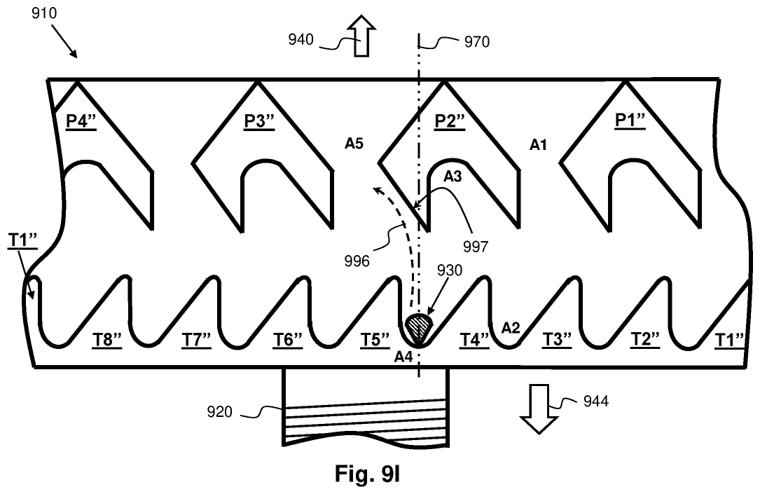

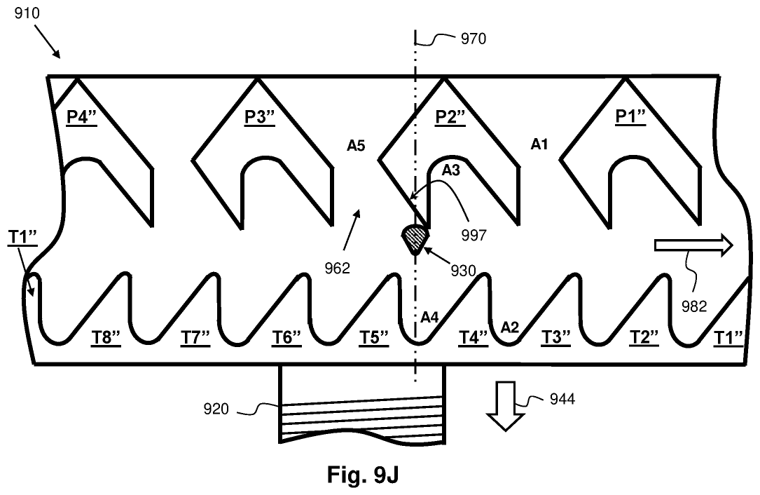

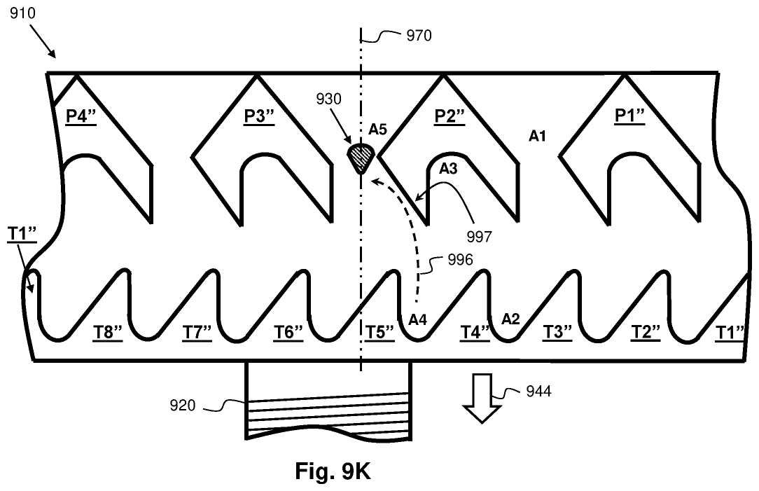

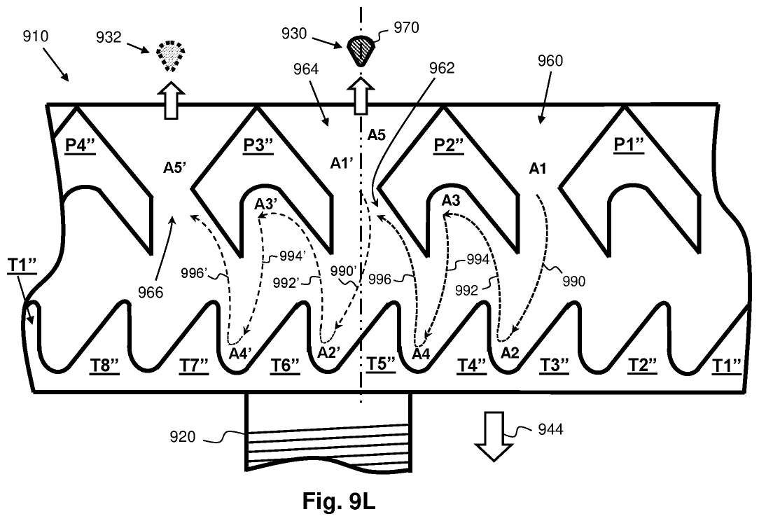

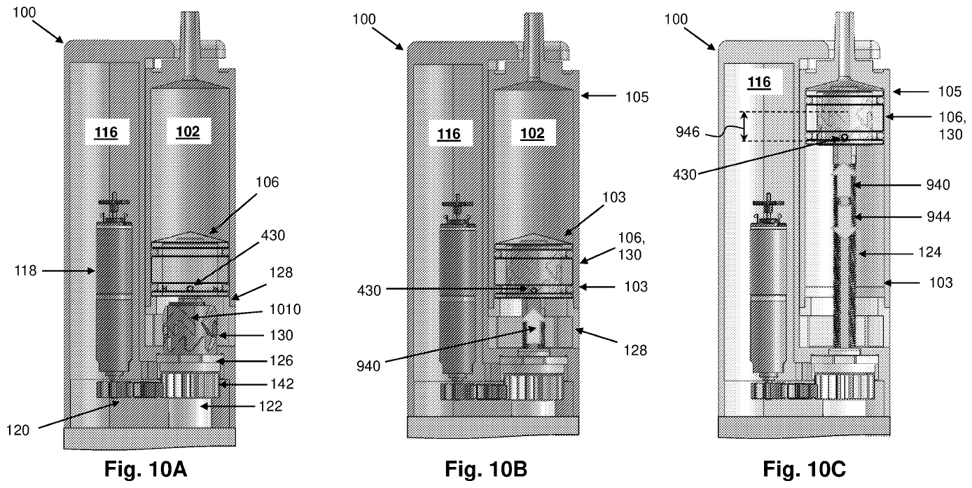

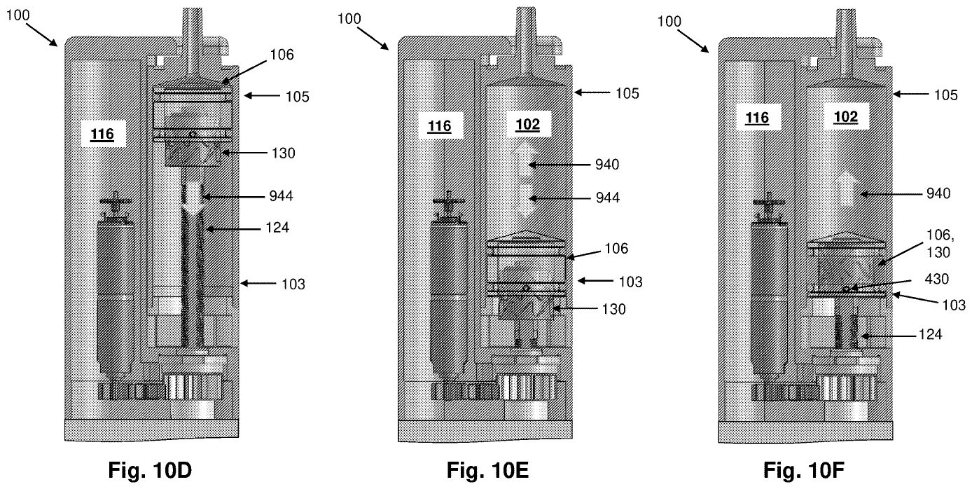

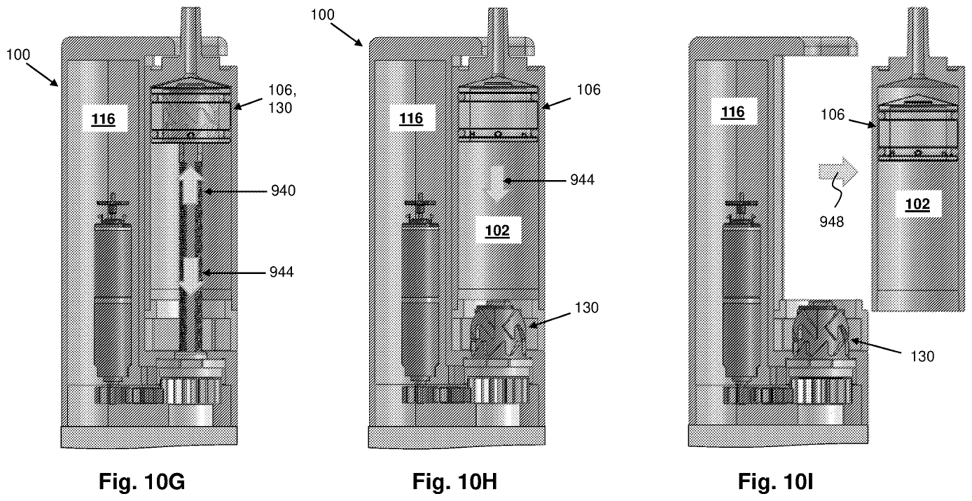

FIGS. 9A-9L show spread-out views of an example cylindrical cam body illustrating, step-by-step, the manner in which the camming surfaces of the cylindrical cam body are engaged with (co-acted by) an example cam pin to effect an alternating pushing and pulling operation of the cylindrical cam body and, consequently, of a plunger head of a syringe. Cylindrical cam body 910 (which in this example is similar to cylindrical cam body 130) is axially locked on the distal end of plunger rod 920 (which in this example is similar to plunger rod 124) but is bi-directionally rotatable about the plunger rod. Pulling members P1″, P2″, P3″ and P4″ are respectively similar to pulling members P1, P2, P3 and P4 of cylindrical cam body 130. Reference line 970 indicates the stationary angular position of cam pin 930. As relative to cylindrical cam body 910.

FIG. 9A shows cylindrical cam body 910 prior to engagement with cam pin 930 (which in this example is similar to cam pins 430, 440, 450 and 460). Cam body 910 provides a ‘five-position’ cycle that includes rotation of cylindrical cam body 910 from the engagement angular position (position A1), in which cam pin 930 abuts entry funnel 960, to the disengagement angular position (position A5), in which cam pin 930 abuts exit funnel 962 of a through channel that is subsequent to the through channel including entry funnel 960. Rotation of cylindrical cam body 910 from engagement angular position Al to the disengagement angular position A5 is done via a contiguous series of alternating pushing-pulling angular positions upon reciprocating axial movement (942) of plunger rod 920 as described herein, for example, hereinafter The series of alternating pushing-pulling angular positions includes, in this example, two pushing angular positions (angular positions A2 and A4) and one pulling angular position (angular position A3) that angularly follows pushing angular position A2 and angularly preceding pushing angular position A4.

Referring to FIG. 9A, when plunger rod 920 moves cylindrical cam body 910 forward (in direction 940) into the plunger head (the plunger head is not shown in FIG. 9A), this movement causes stationary cam pin 930 to enter (950) entry funnel 960 that is formed by, and between, pulling members P1″ and P2″. As cylindrical cam body 910 continues to move in direction 940, cam pin 930 continues its entry in cylindrical cam body 910 through entry funnel 960 until anterior cam surface 980 of pulling member P1″ cammingly engages cam pin 930. Reference numbers 990, 992, 994 and 996 denote successive legs of the zig-zag channel through which cam pin 930 travels as plunger rod 920 reciprocally moves (942) along longitudinal axis 107 (e.g., up and down in FIGS. 9A-9L).

FIG. 9B shows anterior cam surface 980 cammingly engaged by cam pin 930. As cylindrical cam body 910 continues to move further in direction 940, cam surface 980 cammingly co-acts with cam pin 930 to rotate cylindrical cam body 910 on plunger rod 920 in direction 982, about rotational axis 107, until cylindrical cam body 910 reaches the engagement angular position Al, as shown in FIG. 9C. As cylindrical cam body 910 continues to move further in direction 940, cam pin 930 continues its entry deeper into cylindrical cam body 910 from engagement angular position A1 towards pushing angular position A2 along leg 990 of the zig-zag channel. More specifically, to get cam pin 930 to pushing angular position A2 cylindrical cam body 910 is first moved in direction 940 (which is equivale to moving down cam pin 930 relative to cam body 910 along reference line 970) until cam surface 984 of tooth T3″ is cammingly engaged by cam pin 930, as shown in FIG. 9D. (FIG. 9D shows cam surface 984 of tooth T3″ cammingly engaged by cam pin 930.) Then (referring to FIG. 9D), further moving cylindrical cam body 910 forward, in direction 940, causes cam surface 984 to cammingly co-act with cam pin 930 to rotate cylindrical cam body 910 further in direction 982 until cylindrical cam body 910 reaches pushing angular position A2, as shown in FIG. 9E.

Referring again to FIG. 9B, longitudinal (axial) spacing 946 between the pulling recesses and the pushing recesses is an axial distance that cylindrical cam body 910 has to travel in order for the cam pins to switch from engaging the pushing recesses to engaging the pulling recesses, and vice versa. Since the plunger rod is an integral part of the reusable part of the pump device, the exact axial position of the plunger rod and the spacing between the pulling, recesses and the pushing recesses (spacing 946) are always known to the controller of the pump device. So, with spacing 946 being a known factor to the controller of the pump device, the controller always ‘knows’ the exact axial position of plunger head 106 in syringe housing 102 As a result of this, the pump device can accurately control the medicament flow rate, as well as the medicament dosage that is to be delivered to the subject using the pump device.

Referring again to FIG. 9B, line 928 is parallel to longitudinal axis 107. Median line 934 of tooth T8″, hence the entire tooth, slants by an angle a relative to line 928. By way of example the value of angle a may be between 30°and 50°(e.g., α=45°). All teeth of the serrated crown slant by the same angle and to the same direction in the spread-out views. (Teeth T1÷T8 in FIGS. 9A-9L slant about 30° to the right in the spread-out views.)

Referring to FIG. 9E, when cam pin 930 is at angular position A2, movement of plunger rod 920 forward (in direction 940) causes the pushing recess associated with angular position A2 to push cylindrical cam body 910, hence the plunger head (106, FIGS. 1A, 2), forward in the syringe, for example from proximal end 103 of syringe housing 102 (FIG. 1A) to distal end 105 of syringe housing 102 (FIG. 1A), for example to expel air from the syringe housing in preparation for filling the syringe with medicament. After the plunger head reaches the distal end of the syringe housing, or any time before it reaches the distal end of the syringe housing, the direction of the axial movement of plunger rod 920 may be reversed, namely, it can be moved (e.g., by electric motor 118) rearwardly, in direction 944.

Referring to FIG. 9E, as cylindrical cam body 910 is moved rearwardly (in direction 944) by plunger rod 920, cam pin 930 continues to ‘travel’ in cylindrical cam body 910 from pushing angular position A2 towards pulling angular position A3 along leg 992 of the zig-zag, channel. More specifically, to get cam pin 930 to pulling angular position A3 cylindrical cam body 910 is first moved in direction 944 (which is equivale to moving up cam pin 930 relative to cam body 910 along reference line 970) until cam surface 986 of pulling members P2″ is cammingly engaged by cam pin 930, as shown in FIG. 9F. (FIG. 9F shows cam surface 986 of pulling members P2″ cammingly engaged by cam pin 930.) Then (referring to FIG. 9F), further moving cylindrical cam body 910 rearwardly, in direction 944, causes cam surface 986 to cammingly co-act with cam pin 930 to rotate cylindrical cam body 910 again in direction 982 until cylindrical cam body 910 reaches pulling angular position A3, as shown in FIG. 9G.

Referring to FIG. 9G, when cam pin 930 is at angular position A3, movement of plunger rod 920 rearwardly (in direction 944) causes the pulling recess associated with angular position A3 to pull cylindrical cam body 910, hence the plunger head (106, FIGS. 1A, 2), rearwardly in the syringe, for example from distal end 105 of syringe housing 102 (FIG. 1A) to proximal end 103 of syringe housing 102 (FIG. 1A) to, for example to fill the syringe housing with medicament. After the plunger head reaches the proximal end of the syringe housing, or any time before it reaches the proximal end of the syringe housing, the direction of the axial movement of plunger rod 920 may be reversed, namely, it can be moved (e.g., by electric motor 118) forward again, in direction 940.

Referring to FIG. 9G, as cylindrical cam body 910 is moved again in direction 940, cam pin 930 continues its travel in cylindrical cam body 910 from pulling angular position A3 towards pushing angular position A4 along leg 994 of the zig-zag channel. More specifically, to get cam pin 930 to pushing angular position A4 cylindrical cam body 910 is moved in direction 940 (which is equivale to moving cam pin 930 down relative to cam body 910 along reference line 970) until cam surface 988 of tooth T4″ is cammingly engaged by cam pin 930, as shown in FIG. 9H (FIG. 9H shows cam surface 988 of tooth T4″ cammingly engaged by cam pin 930.) Then (referring to FIG. 9H), further moving cylindrical cam body 910 forward (in direction 940) causes cam surface 988 to cammingly co-act with cam pin 930 to rotate cylindrical cam body 910 again in direction 982 until cylindrical cam body 910 reaches pushing, angular position A4, as shown in FIG. 9I.

Referring to FIG. 9I, when cam pin 930 is at angular position A4, movement of plunger rod 920 forward (in direction 940) again causes the pushing recess associated with angular position A4 to push cylindrical cam body 910, hence the plunger head (106, FIGS. 1A, 2), forward in the syringe, for example from proximal end 103 of syringe housing 102 (FIG. 1A) to distal end 105 of syringe housing 102 (FIG. 1A), for example to deliver the medicament the syringe housing was filled with to a patient. After the plunger head reaches the distal end of the syringe housing, or any time before it reaches the distal end of the syringe housing, the direction of the axial movement of plunger rod 920 may be reversed, namely, it can be moved (e.g., by electric motor 118) rearwardly, in direction 944 to release plunger rod 920 from the plunger head, for example for replacing a used (empty) disposable syringe (102) by a new disposable syringe.

As cylindrical cam body 910 is moved rearwardly (in direction 944), cam pin 930 continues its relative travel from push angular position A4 towards disengagement angular position A5 along leg 996 of the zig-zag channel, through (it is guided by) exit funnel 962. More specifically, to get cam pin 930 to disengagement angular position A5 cylindrical cam body 910 is axially moved in direction 944 (which is equivale to moving cam pin 930 up relative to cam body 910 and along reference line 970) until cam surface 997 of pulling member P2″ is cammingly engaged by cam pin 930, as shown in FIG. 9J. (FIG. 9J shows cam surface 997 of pulling member P2″ cammingly engaged by cam pin 930.) Then (referring to FIG. 9J), further moving cylindrical cam body 910 rearwardly, in direction 944, causes cam surface 997 to cammingly co-act with cam pin 930 to rotate cylindrical cam body 910 again about rotational axis 107, in direction 982, until cylindrical cam body 910 reaches disengagement angular position A5, as shown in FIG. 9K. Referring to FIG. 9K, when cam pin 930 is at disengagement angular position A5, further movement of plunger rod 920 rearwardly (in direction 944) disengages cylindrical cam body 910 from cam pin 930, as shown in FIG. 9L.

Anterior cam surface 980 of pulling member P1″, cam surface 984 of tooth T3″, posterior cam surface 986 of pulling member P2″, cam surface 988 of tooth T4″ and posterior cam surface 997 of pulling member P2″ slant in a way that reciprocating axial movement (942, FIG. 9A) of plunger rod 920 causes these cam surfaces to successively co-act (i.e., first anterior cam surface 980, then cam surface 984, and so on) with cam pin 930 to unidirectionally rotate cylindrical cam body 910 in direction 576. Cam pin 930 is one cam pin that is described as unidirectionally rotating cylindrical cam body 910 in direction 576. However, as described herein and shown, for example, in FIG. 4, a plunger head includes more than one cam pin, and all of the cam pins operate in unison to unidirectionally rotate cylindrical cam body 910 in direction 576. In-unison operation of the cam pins also means that all of the cam pins enter (engage) the cylindrical cam body at the same time through different through channels (through their entry funnels), then they travel through similar zig-zag legs and, finally, exit (disengaged from) the cylindrical cam body at the same time through different through channels (through their exit funnels). An entry through channel of a particular cam point is an exit through channel of another cam point. Similarly, an exit through channel of a particular cam point is an entry through channel of another cam point.

FIG. 9L also shows two cam pins (out of a total of four cam pins), one is shown at 930 and the other at 932. When cam pin 930 rotates cylindrical cam body 910 to enter cylindrical cam body 910 through entry funnel 960 at the engagement angular position (A1), cam pin 932 simultaneously rotates cylindrical cam body 910 to enter cylindrical cam body 910 through entry funnel 964 at the same time (as do the other cam pins, though through other entry funnels) at its engagement angular position (A1′). The other cam pins simultaneously travel via similar paths to their engagement angular positions. This way, all cam pins enter cylindrical cam body 910 simultaneously.

When cam pin 930 rotates cylindrical cam body 910 to its first pushing angular position (A2) while traveling to this position via leg 990 of the guiding zig-zag channel, cam pin 932 cammingly rotates cylindrical cam body 910 to its first pushing angular position (A2′) while traveling to this position via leg 990′of the zig-zag channel. The other cam pins simultaneously travel (zig-zag) via similar paths to their pushing angular positions. This way, all cam pins can be pushed simultaneously by cylindrical cam body 910, to thereby push the plunger head 106.

When cam pin 930 rotates cylindrical cam body 910 to its pulling angular position (A3) while traveling to this position via leg 992 of the zig-zag path, cam pin 932 rotates cylindrical cam body 910 to its pulling angular position (A3′) while traveling to this position via leg 992′ of the zig-zag channel. The other two cam pins simultaneously travel via similar paths to their pulling angular positions. This way, all cam pins can be pulled simultaneously by cylindrical cam body 910, to thereby pull the plunger head 106.

When cam pin 930 rotates cylindrical cam body 910 to its second pushing angular position (A4) while traveling to this position via leg 994 of the guiding zig-zag channel, cam pin 932 rotates cylindrical cam body 910 to its second pushing angular position (A4′) while traveling to this position via leg 994′of the zig-zag channel. The other two cam pins simultaneously travel via similar paths to their pushing angular positions. This way, all cam pins can be pushed simultaneously by cylindrical cam body 910, to thereby push the plunger head 106 a second time.

When cam pin 930 rotates cylindrical cam body 910 to its disengagement angular position AS while traveling to this position via leg 996 of the zig-zag channel, cam pin 932 rotates cylindrical cam body 910 to its disengagement angular position A5′ while traveling to this position via leg 996′of the zig-zag channel. The other cam pins simultaneously travel via similar paths to their disengagement angular positions. This way, all cam pins can be simultaneously disengaged from cylindrical cam body 910 to enable releasing of plunger rod 910 from plunger head 106. So, when cam pin 930 enters cylindrical cam body 910 through entry funnel 960, travels along the zig-zag channel including legs 990, 992 994 and 998 and exists cylindrical cam body 910 via exit funnel 962, all other the cam pins simultaneously follow suit. For example, cam pin 932 enters cylindrical cam body 910 through entry funnel 964, travels along a similar zig-zag channel including legs 990′, 992′, 994′and 996′and, then, exits cylindrical cam body 910 via exit funnel 966.

Referring again to FIG. 9A, pulling member P3″ includes first (external) stop surface 912 and tooth T6″ includes stop surface 914. Stop surfaces 912 and 914 are laterally spaced apart (916) such that stop surface 912 precedes stop surface 914 relative to the rotation direction (576) of cylindrical cam body 910. Space 916 is selected such that a cam pin entering cylindrical cam body 910 via the through channel between pulling members P2″ and P3″ will be able to engage, and cammingly co-act, with the cam surface of tooth T5″ Space 916 is also selected such that a cam pin residing in the pushing recess between teeth T5″ and T6″ will be able to engage, and cammingly co-act, with the internal cam surface 924 of pulling member P3″.

Similarly, pulling member P3″ includes a second (internal) stop surface 918 and tooth T7″ includes stop surface 920. Stop surfaces 918 and 920 are laterally spaced apart (922) such that stop surface 918 precedes stop surface 920 relative to the rotation direction (576) of cylindrical cam body 910. Space 922, which may be identical to space 916, is selected such that a cam pin residing in the pulling recess of pulling member P3″ will be able to engage, and cammingly co-act, with the cam surface of tooth T6″. Space 922 is also selected such that the cam pin will be able to engage, and cammingly co-act, with external cam surface 926 of pulling member P3″. Similarly, spacings that are identical to spacings 916 and 922 exist between the two stop surfaces of each pulling member Pᵢ″ and the stop surfaces of the respective teeth. The spacing between the stop surfaces enables smooth passage of all cam pins in the guiding zig-zag channel (e.g., guiding zig-zag channel 710).

By way of example, pump device 100 is a four-stroke cycle pump implementing the following four distinct plunger rod (hence plunger head) strokes:
1. “Engaging and air Expelling” stroke (the 1st stroke): plunger rod 124 is linearly moved, for example by electric motor 118, out of the stow (home) position in reusable part 116 of pump device 100 to engage the plunger head 106 at the bottom (proximal end 103) of syringe housing 102, and continually pushes plunger bead 106 to distal end 105 of syringe housing 102 to empty syringe 101 from air (for example);
2. “Full” stroke (the 2nd stroke): plunger rod 124 is linearly moved from distal end 105 of syringe housing 102 back to the bottom (proximal end 103) of syringe housing 102 to fill syringe housing 102 with medicament (for example);
3. “Delivery” stroke (the 3rd stroke): plunger rod 124 is linearly moved from proximal end 103 of syringe housing 102 back to distal end 105 of syringe housing 102 to expel medicament from syringe housing 102 (for example); and
4. “Disengaging and Stowing” stroke (the 4th stroke): plunger rod 124 is linearly moved from distal end 105 of syringe housing 102 rearwardly to such that it is disengaged from plunger head 106 and continually moves all the way backwards to the stowing position in reusable part 116 of pump device 100.

So, plunger rod 124 (hence plunger head 106) makes four complete passes through syringe 101 to complete one full operating cycle of pump device 100 from engagement of plunger rod 124 with plunger head 106, to the disengagement of plunger rod 124 from plunger head 106. “Stroke”, as used herein, is the maximal axial distance that can be travelled by the plunger rod (hence the cylindrical cam body and plunger head) as the plunger rod moves in the cylindrical syringe housing between one predetermined axial position along the longitudinal axis of the cylindrical syringe housing, to another. In the example shown in FIGS. 10A-10I, a stroke distance of plunger rod 124 may be the distance traveled by plunger rod 124 between proximal end 103 of syringe housing 102 (or the stowing/home position of the cylindrical cam body 130) and distal end 105 of syringe housing 102. Depending on the use of the pump device, an operating cycle of the pump device may include less than four strokes or more than four strokes, and the number of serrated crown teeth (hence the number of pushing recesses), the number of pulling members, and the number of pulling recesses per pulling member can be set to adhere to the selected operating cycle of the pump device.

FIGS. 10A-10I show an example nine-step process for operating pump device 100 according to an example embodiment (Some of the nine steps may be unified into a single step.) The nine-step process generally includes operationally engaging disposable syringe 101 with reusable part 116 of pump device 100, engaging plunger rod 124 of pump device 100 with plunger head 106 of the disposable syringe 101, operating the pump device while the disposable syringe and the reusable part of the pump device are engaged, and, finally, disengaging plunger rod 124 from plunger head 106 to enable decoupling of disposable syringe 101 from reusable part 116 of the pump device. The nine-step operation process of the pump device demonstrates (includes) the four-stroke operating cycle of pump device 100.

FIG. 10A shows the first step of the nine-step process of operating the pump device. (This step of the nine-step process of operating the pump device corresponds to FIG. 9A, which shows a cylindrical cam body before it engages the plunger head.) In this step, syringe 101 is attached to reusable part 116 of pump device 100, and cylindrical cam body 130 is at stow/home position within reusable part 116 of pump device 100, disengaged from plunger head 106.

FIG. 10B shows the second step of the nine-step process of operating the pump device. (This step of the nine-step process of operating the pump device corresponds to FIG. 9E, which shows a cylindrical cam body that has cammingly (and rotatably) aligned itself, by co-acting with the cam pins, to a first pushing angular position in the plunger head.) In this step, plunger rod 124 is axially (longitudinally) partly extended forward, in direction 940, to engage (lock onto) plunger head 106 by engaging cylindrical cam body 130 with the cam pins of plunger head. One of the four cam pins (e.g., cam pin 430) is shown resting in the first pushing recess in cylindrical cam body 130, which corresponds to the first pushing angular position of cylindrical cam body 130. At this stage plunger rod 124 is at proximal end 103 of syringe 101. ready to, for example, expel air from syringe 101.

FIG. 10C shows the third step of the nine-step process of operating the pump device. (This step of the nine-step process of operating the pump device also corresponds to FIG. 9E, which shows the cylindrical cam body still in its pushing angular position in the plunger head.) In this step, plunger rod 124 is fully extended in syringe 101 (cam body 130 is at distal end 105 of syringe 101), after having travelled forward the first stroke in syringe 101 (e.g., from proximal end 103 of syringe 101 to distal end 105 of syringe 101) to expel air (for example) from syringe 101. At this stage, plunger rod 124 is almost ready for its second stroke to, for example, fill syringe 101 with medicament by moving plunger rod 124, hence plunger head 106, rearwardly (backwards), in direction 944 To start moving plunger head 106 backwards, in direction 944, by plunger rod 124, plunger rod 124 has first to be moved rearwardly (944) by the distance (distance 946) required to start guiding cam pin 430 into the pulling recesses of cam body 130 such that cylindrical cam body 130, by cammingly co-acting with the cam pins, rotatably aligns itself to the pulling angular position in plunger head 106.

FIG. 10D shows the fourth step of the nine-step process of operating the pump device. (This step of the nine-step process of operating the pump device corresponds to FIG. 9G, which shows a cylindrical cam body that has cammingly (and rotatably) aligned itself, by co-acting with the cam pins, to a pulling angular position in the plunger head ) In this step, plunger head 106 is ready to be moved by plunger rod 124 rearwardly, in direction 944 (from distal end 105 of syringe 101 to proximal end 103 of syringe 101), to complete the second stroke (e.g., ‘filling’ stroke) of plunger rod 124, to pull back plunger head 106, for example, to fill syringe 101 with medicament.

FIG. 10E shows the fifth step of the nine-step process of operating the pump device. (This step of the nine-step process of operating the pump device also corresponds to FIG. 9G, which shows the cylindrical cam body still in the pulling angular position in the plunger head.) FIG. 10E shows plunger rod 124 after having completed the second stroke (e.g., ‘filling’ stroke). At this stage, plunger rod 124 is almost ready for its third stroke (e.g., ‘medicament delivery’ stroke) to, for example, expel medicament from syringe 101, or to prime an infusion set (tubing, system), by moving plunger rod 124, hence plunger head 106, forward, in direction 940. To start moving plunger head 106 forward (in direction 940) by plunger rod 124, plunger rod 124 has first to be moved forward (940) by the distance (distance 946) required to start guiding cam pin 430 into the second (subsequent) pushing recess of cam body 130 such that cylindrical cam body 130, by cammingly co-acting with the cam pins, rotatably aligns itself to the subsequent pushing angular position in plunger head 106.

FIG. 10F shows the sixth step of the nine-step process of operating the pump device. (This step of the nine-step process of operating the pump device corresponds to FIG. 9E, which shows a cylindrical cam body that has cammingly (and rotatably) aligned itself, by co-acting with the cam pins, to a pushing angular position in the plunger head. ) One of the four cam pins (e.g., cam pin 430) is shown in FIG. 10F resting in a second (subsequent) pushing recess in cylindrical cam body 130, which corresponds to a second pushing angular position of cylindrical cam body 130. In this stage, plunger head 106 is still engaged with cam body 130 and ready to be moved by plunger rod 124 forward, in direction 940, to start the third stroke (e.g., ‘filling’, ‘priming’, etc.) of plunger rod 124 to expel medicament from syringe 101, or to prime an infusion set.

FIG. 10G shows the seventh step of the nine-step process of operating the pump device. (This step of the nine-step process of operating the pump device also corresponds to FIG. 9E, which shows the cylindrical cam body still in its pushing angular position in the plunger head.) In this stage, plunger rod 124 is fully extended in syringe 101 (cam body 130 is at distal end 105 of syringe 101) after having completed the third stroke (e.g., ‘medicament delivery’ stroke) in syringe 101 by moving from proximal end 103 of syringe 101 to distal end 105 of syringe 101 to deliver medicament (for example) from syringe 101.

After having travelled the third stroke, plunger rod 124 is ready for disengagement from plunger head 106 and for stowing in the pump device. To disengage plunger rod 124 from plunger head 106, plunger rod 124 is moved rearwardly, in direction 944 Moving plunger rod 124 in direction 944 causes cylindrical cam body 130 to cammingly co-act with the cam pins of plunger head 106 to rotate cylindrical cam body 130 such that cylindrical cam body 130 rotatably aligns itself in the disengagement angular position in plunger head 106, similarly to what FIG. 9K shows. Once cylindrical cam body 130 is rotatably aligned in the disengagement angular position in plunger head 106, further moving plunger rod 124 rearwardly, in direction 944, disengages plunger rod 124 from plunger head 106, similarly to what FIG. 9L shows.

FIG. 10H shows the eighth step of the nine-step process of operating the pump device. (This step of the nine-step process of operating the pump device corresponds to FIG. 9L, which shows the cylindrical cam body disengaged from an example cam pin of a plunger head.) At this stage, plunger rod 124 is fully retrieved and back in the stowing position in reusable part 116 of pump device 100 after having completed the fourth stroke (e.g., ‘stowing’stroke).

FIG. 10I shows the ninth step of the nine-step process of operating the pump device, which is, or includes, removing (948) syringe 101 from reusable part 116 while plunger rod 124 is in the stowing/home position in reusable part 116. If required or desired, the removed syringe can be replaced by a new, similar, disposable syringe.

The benefits of the pump device of the present invention comparing to conventional pumps are at least the following:
1. Flexibility to design a quick release camming mechanism for a desired need, including the ability to engage the plunger rod with the plunger head by simply moving the plunger rod forward, and to disengage the plunger rod from the plunger head by simply moving the plunger rod rearwardly;
2. Reduced cost of goods (COGS) related to disposable part (syringe) of a pump device;
3. Fewer parts that are inseparately embedded in the disposable part (syringe);
4. Improved product sustainability;
5. Higher precision propulsion system due to the plunger rod (a leadscrew) being part of the reusable part of the pump device, which enables operating it with higher precision and smaller, or tightened, tolerances;
6. Ability to design a rounded syringe, as opposed to oval syringe or syringes with other shapes;
7. Simplified assembly process of the disposable part (syringe);
8. Supporting, or promoting, pre-filled reservoir concept;
9. Higher leadscrew positioning and position monitoring accuracy;
10. More efficient occlusion control due to enhanced control of the position of the lead screw (plunger rod), and
11. Improved medicament transfer system concept.

The articles “a” and “an” are used herein to refer to one or to more than one (e.g., to at least one) of the grammatical object of the article, depending on the context. By way of example, depending on the context, “an element” can mean one element or more than one element. The term “including” is used herein to mean, and is used interchangeably with, the phrase “including but not limited to”. The terms “or” and “and” are used herein to mean, and are used interchangeably with, the term “and/or,” unless context clearly indicates otherwise. The term “such as” is used herein to mean, and is used interchangeably, with the phrase “such as but not limited to”.

Having thus described exemplary embodiments of the invention, it will be apparent to those skilled in the art that modifications of the disclosed embodiments will be within the scope of the invention. Alternative embodiments may, accordingly, include functionally equivalent objects/articles. For example, the plunger head and/or the plunger rod and/or the cylindrical cam body may have a different design (e.g., different shape, size and/or material, different numbers of cam pins, different numbers of cam surfaces and/or stop surfaces, different numbers of pulling recesses, different numbers of pushing recesses, etc.) than the ones described herein and shown in the drawings. Features of certain embodiments may be used with other embodiments shown herein. The present disclosure is described in connection with pump devices that include a disposable reservoir (syringe) and a reusable part. However, the present disclosure may be relevant to (e.g., it may be implemented by, used with or for) other types of ‘two-part’ devices, pumps, syringes, therapeutic drug dispensing devices, and the like. Hence the scope of the claims that follow is not necessarily limited by the disclosure herein.

1. A quick connecting-release camming mechanism for a pump device including a reusable part and a disposable part, the camming mechanism comprising:
a rotatable cam body comprising a plurality of cam surfaces, said rotatable cam body axially affixed to a distal end of a plunger rod and rotatable about said plunger rod, said plunger rod moveable between a stowing position in the reusable part and an extended position; and
a plurality of cam pins residing in a plunger head of the disposable reservoir, said plunger head bi-directionally linearly moveable in the disposable reservoir,
wherein the plurality of cam surfaces and the plurality of cam pins configured to cammingly co-act to rotate the rotatable cam body in, and relative to, the plunger head to and from an engagement angular position in which the cam body and the plunger head are engaged to a disengagement angular position in which the cam body and the plunger head are disengaged, via a series of alternating push-pull angular positions for alternately pushing and pulling the plunger head in the disposable part, and wherein rotating the rotatable cam body from one angular position to another is effected by axially reciprocating the plunger rod relative to the plunger head.

2. A mechanism for selecting an operational interaction between an axially reciprocating plunger rod of a pump device and a plunger head bidirectionally moveable between a first axial position and a second axial position in a syringe and restrained in the syringe against rotational movement, the mechanism comprising:
a cylindrical cam body axially restrained on and rotatable about the axially reciprocating plunger rod and receivable by a one-sided open chamber concentrically formed in the plunger head by a cylindrical wall, said cylindrical cam body comprising a plurality of spaced cam surfaces circumferentially located on the cylindrical cam body; and
a plurality of cam pins equiangularly protruding from the cylindrical wall towards a longitudinal axis of the plunger head, the plurality of cam pins configured to co-act with the cylindrical cam body on reciprocal movement of the plunger rod to sequentially traverse the plurality of cam surfaces to rotate the cylindrical cam body in, and relative to, the chamber to successively different angular positions corresponding to different operational interactions between the plunger rod and the plunger head.

3. The mechanism as in claim 2, wherein the plurality of cam pins co-act with the cam body upon axial reciprocal movement of the cam body in the chamber to sequentially traverse the plurality of cam surfaces to unidirectionally rotate the cam body relative to the chamber to successively different rotative operational position on successive operation of the reciprocating plunger rod.

4. The mechanism as in claim 2, wherein the different operational interactions between the plunger rod and the plunger head include:
(i) engaging the plunger rod with the plunger head upon axial movement of the cam body, by the plunger rod, into the chamber;
(ii) alternately pushing and pulling the plunger head in the syringe by the plunger rod upon axial reciprocating movement of the plunger rod; and
(iii) disengaging the plunger rod from the plunger head upon axial movement of the cam body, by the plunger rod, out of the chamber.

5. The mechanism as in claim 2, wherein the successively different angular positions of the cam body in the chamber comprise:
an engagement angular position, in which the plunger rod is engaged with the plunger head by axially moving the cam surfaces of the cam body forward by the plunger rod against the cam pins;
a disengagement angular position, in which the cam body is releasable from the cam pins by axially moving the cam body rearwardly from the chamber by the plunger rod, and
a series of alternating pushing-pulling angular positions between the engagement angular position and the disengagement angular position, in which the cam body alternately pushes and pulls the plunger head in the syringe on successive axial reciprocating movement of the plunger rod.

6. The mechanism as in claim 5, wherein the series of alternating pushing-pulling angular positions comprises:
(i) a first angular position, in which the cam body is in position in the plunger head to push the plunger head forward from the first axial position in the syringe to the second axial position in the syringe;
(ii) a second angular position subsequent to the first angular position, in which cam body is in position in the plunger head to pull the plunger head rearwardly from the second axial position in the syringe to the first axial position in the syringe; and
(iii) a third angular position subsequent to the second angular position, in which the cam body is, again, in position to push the plunger head forward from the first axial position in the syringe to the second axial position in the syringe.

7. The mechanism as in claim 6, wherein the first angular position is for expelling air from the syringe, the second angular position is for filling the syringe with medicament, and the third angular position is for expelling the medicament from the syringe.

8. The mechanism as in claim 5, wherein the cylindrical cam body comprises:
a number ‘n’ of circumferential teeth equiangularly spaced apart on a proximal end of the cam body and forming ‘n’ U-shaped pushing recesses to axially push the cam pins, hence the plunger head in the syringe; and
a number ‘n/2’ of circumferential pulling members, the pulling members equiangularly spaced apart on the distal end of the cam body, each pulling member comprising a U-shaped pulling recess to axially pull the cam pins, hence the plunger head in the syringe.

9. The mechanism as in claim 8, wherein each U-shaped pushing recess generally opens towards a distal end of the cam body at an acute angle with respect to a line parallel to a longitudinal axis of the cam body, and each U-shaped pulling recess generally opens towards a proximal end of the cam body at an acute angle with respect to the line parallel to the longitudinal axis of the cam body.

10. The mechanism as in claim 8, wherein the ‘n’ teeth and the ‘n/2’ pulling members are disposed on the cylindrical cam body in alternating relation such that every odd tooth (Ti) abuts a through channel that is jointly formed by and between two adjacent pulling members (Pi, Pi+1), and every even tooth (Ti+1) abuts a “U”-shaped pulling recess of a particular pulling member (Pi+1).

11. The mechanism as in claim 10, wherein the through channel formed by and between each two adjacent pulling members comprises.
an entrance funnel, which is configured to rotatively receive and guide a respective one of the plurality of cam pins into the engagement angular position upon insertion of the cylindrical cam body into the chamber; and
an exit funnel, which is configured to rotatively receive and guide a respective one of the plurality of cam pins out of the disengagement angular position upon retraction of the cam body from the chamber,
wherein the entrance funnel and the exit funnel of each through channel are lengthwise positioned ‘back-to-back’ such that a narrow opening of the entrance funnel coincides with a narrow opening of the exit funnel.

12. The mechanism as in claim 11, wherein the entrance funnel is formed by an axially anterior pair of conjugated cam surfaces comprising an anterior cam surface of a particular pulling member (Pi) and an anterior cam surface of a pulling member (Pi+1) adjacent to the particular pulling member (Pi), and
wherein the exit funnel is formed by an axially posterior pair of conjugated surfaces comprising a posterior cam surface of a particular pulling member (Pi) and a posterior surface of an adjacent pulling member (Pi+1).

13. The mechanism as in claim 12, wherein the number of the through channels is identical to the number of the cam pins, and wherein each cam pin is configured to abut an entrance funnel of a particular one of the through channels for insertion of the cam body into the chamber of the plunger head, and to abut an exit funnel of a different one of the through channels for extraction of the cam body from the chamber of the plunger head.

14. The mechanism as in claim 13, wherein n=8, and the number of the cam pins is four.

15. The mechanism as in claim 8, wherein the ‘n’ circumferential teeth form a serrated crown in the proximal end of the cam body, wherein each tooth of the serrated crown is a right-angled triangle shaped tooth comprising a cam surface of the plurality of cam surfaces and a stop surface, and wherein each particular “U”-shaped pushing recess is formed by connecting (‘bridging’) the cam surface of a particular tooth (Ti) and the stop surface of a subsequent tooth (Ti+1) by a bottom surface, and
wherein each particular pulling member (Pi) comprises a first prong and a second prong, the first prong comprising a cam surface of the plurality of cam surfaces and the second prong comprises a stop surface, and wherein a “U”-shaped pulling recess in the particular pulling member (Pi) is formed by connecting (‘bridging’) the cam surface and the stop surface of the particular pulling member (Pi) by a bottom surface.

16. The mechanism as in claim 15, wherein the ‘n/2’ pulling members are axially distanced away from the serrated crown and define, therewith, a guiding ‘zig-zag’ channel for traversal by the plurality of cam pins upon rotation of the cylindrical cam body from the engagement angular position to the disengagement angular position via the series of alternating pushing-pulling angular positions upon reciprocating movement of the plunger rod.

17. The mechanism as in claim 15, wherein the serrated crown comprises a ring-shaped base at the proximal end of the cam barrel, and the circumferential teeth longitudinally extend from the ring-shaped base at a right angle relative to a plane of the ring-shaped base such that all teeth point to the distal end of the cam body,
wherein, for each tooth, the cam surface is slanting with respect to a line parallel to the longitudinal axis of the cam body, and the stop surface is parallel to the line parallel to the longitudinal axis of the cam body.

18. A pump device for delivering medicament, comprising:
a disposable reservoir comprising:
a plunger head bidirectionally moveable in the disposable reservoir between a first axial position and a second axial position, said plunger head comprising:
a one-sided open chamber concentrically formed in the plunger head by a cylindrical wall, and
a plurality of cam pins equiangularly and radially protruding from the cylindrical wall to a longitudinal axis of the plunger head; and
a reusable part comprising:
a plunger rod configured for axial reciprocating movement, and
a cylindrical cam body axially restrained on and rotatable about the plunger rod and receivable by the one-sided open chamber, said cylindrical cam body comprising a plurality of cam surfaces circumferentially located on the cylindrical cam body,
wherein the plurality of cam pins configured to co-act with the cylindrical cam body on axial reciprocating movement of the plunger rod to sequentially traverse the plurality of cam surfaces to rotate the cylindrical cam body in, and relative to, the one-sided open chamber to successively different angular positions corresponding to different operational interactions between the plunger rod and the plunger head.

19. The mechanism as in claim 18, wherein the cam pins are configured to traverse the plurality of cam surfaces of the cam body on axial reciprocal movement of the cam body in the chamber to unidirectionally rotate the cam body relative to the chamber to successively different rotative operational position on successive operation of the reciprocating plunger rod.

20. A method of operating a pump device having a plunger rod provided with a rotatable cam body, comprising:
engaging a disposable reservoir provided with a plunger head with a reusable part of the pump device and, while the disposable reservoir and the reusable part of the pump device are engaged
axially moving the plunger rod in the disposable reservoir in a reciprocating manner to cause the rotatable cam body to cammingly co-act with cam pins in the plunger head to rotate the rotatable cam body to an engagement angular position in which the plunger rod is engaged with the plunger head, then through a series of alternating pushing-pulling angular positions corresponding to a series of alternating pushing forward and pulling backwards the plunger head in the disposable reservoir, and, then, to a disengagement angular position in which the plunger rod is disengageable from the plunger head.
